SEQUENCE LISTING (33 sequences)

SEQ ID NO: 1 - protein, 28 aa, Artificial
  linker sequence
SERALALYSTHRTHRPRLYSLEGLYGLY

SEQ ID NO: 2 - protein, 48 aa, Artificial
  linker sequence
GLYGLYGLYGLYSERGLYGLYGLYGLYSERGLYGLYGLYGLYSERGLY

SEQ ID NO: 3 - protein, 42 aa, Artificial
  linker sequence
SERALALYSTHRTHRPRLYSLEGLGLGLYGLPHESERGLALA

SEQ ID NO: 4 - DNA, 1817 bp, Artificial
  Plasmid
ccccaggctttacactttatgcttccggctcgtatgttgtgtggaattgtgagcggataacaatttcacacaggaaacagctatgaccatgattacgaatttctgaagaaggagatatacatatgaaatacctattgcctacggcagccgctggcttgctgctgctggcagctcagccggccatggcgcaggtgcaactgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggtcaaggaacctcagtcaccgtctcctcagccaaaacaacacccaagcttgaagaaggtgaattttcagaagcacgcgtagatatcttgctcacccaaactccagcttctttggctgtgtctctagggcagagggccaccatctcctgcaaggccagccaaagtgttgattatgatggtgatagttatttgaactggtaccaacagattccaggacagccacccaaactcctcatctatcatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggaggcaccaagctggaaatcaaacgggctgatgctgcggccgctggtggtggtggttctggcggcggtggtagcggtggtggcggctccggtggtggtggtagccaggtgcagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcctcagccaaaacaacacccaagcttggcggtgatatcgtgctcactcagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgggctgatactgcaccaactggatccgaacaaaagctgatctcagaagaagacctaaactcacatcaccatcaccatcactaatctaga

SEQ ID NO: 5 - protein, 1603 aa, Artificial
  Plasmid
METLYSTYRLELEPRTHRALAALAALAGLYLELELELEALAALAGLNPRALAMETALAGLNVALGLNLEGLNGLNSERGLYALAGLLEALAARGPRGLYALASERVALLYSMETSERCYSLYSALASERGLYTYRTHRPHETHRARGTYRTHRMETHISTRPVALLYSGLNARGPRGLYGLNGLYLEGLTRPILEGLYTYRILEASNPRSERARGGLYTYRTHRASNTYRASNGLNLYSPHELYSASPLYSALATHRLETHRTHRASPLYSSERSERSERTHRALATYRMETGLNLESERSERLETHRSERGLASPSERALAVALTYRTYRCYSALAARGTYRTYRASPASPHISTYRSERLEASPTYRTRPGLYGLNGLYTHRTHRLETHRVALSERSERALALYSTHRTHRPRLYSLEGLGLGLYGLPHESERGLALAARGVALASPILEVALLETHRGLNSERPRALAILEMETSERALASERPRGLYGLLYSVALTHRMETTHRCYSSERALASERSERSERVALSERTYRMETASNTRPTYRGLNGLNLYSSERGLYTHRSERPRLYSARGTRPILETYRASPTHRSERLYSLEALASERGLYVALPRALAHISPHEARGGLYSERGLYSERGLYTHRSERTYRSERLETHRILESERGLYMETGLALAGLASPALAALATHRTYRTYRCYSGLNGLNTRPSERSERASNPRPHETHRPHEGLYSERGLYTHRLYSLEGLILEASNARGALAASPTHRALAALAALAGLYGLYGLYGLYSERGLYGLYGLYGLYSERGLYGLYGLYGLYSERGLYGLYGLYGLYSERGLNVALGLNLEGLNGLNSERGLYALAGLLEVALARGPRGLYSERSERVALLYSILESERCYSLYSALASERGLYTYRALAPHESERSERTYRTRPMETASNTRPVALLYSGLNARGPRGLYGLNGLYLEGLTRPILEGLYGLNILETRPPRGLYASPGLYASPTHRASNTYRASNGLYLYSPHELYSGLYLYSALATHRLETHRALAASPGLSERSERSERTHRALATYRMETGLNLESERSERLEALASERGLASPSERALAVALTYRPHECYSALAARGARGGLTHRTHRTHRVALGLYARGTYRTYRTYRALAMETASPTYRTRPGLYGLNGLYTHRSERVALTHRVALSERSERALALYSTHRTHRPRLYSLEGLYGLYASPILELELETHRGLNTHRPRALASERLEALAVALSERLEGLYGLNARGALATHRILESERCYSLYSALASERGLNSERVALASPTYRASPGLYASPSERTYRLEASNTRPTYRGLNGLNILEPRGLYGLNPRPRLYSLELEILETYRASPALASERASNLEVALSERGLYILEPRPRARGPHESERGLYSERGLYSERGLYTHRASPPHETHRLEASNILEHISPRVALGLLYSVALASPALAALATHRTYRHISCYSGLNGLNSERTHRGLASPPRTRPTHRPHEGLYGLYGLYTHRLYSLEGLILELYSARGALAASPALAALAALAALAGLYSERGLGLNLYSLEILESERGLGLASPLEASNSERHISHISHISHISHISHIS

SEQ ID NO: 6 - DNA, 1817 bp, Artificial
  Plasmid
ccccaggctttacactttatgcttccggctcgtatgttgtgtggaattgtgagcggataacaatttcacacaggaaacagctatgaccatgattacgaatttctgaagaaggagatatacatatgaaatacctattgcctacggcagccgctggcttgctgctgctggcagctcagccggccatggcgcaggtgcaactgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcctcagccaaaacaacacccaagcttgaagaaggtgaattttcagaagcacgcgtagatatcgtgctcactcagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgggctgatactgcggccgctggtggtggtggttctggcggcggtggtagcggtggtggcggctccggtggtggtggtagccaggtgcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggtcaaggaacctcagtcaccgtctcctcagccaaaacaacacccaagcttggcggtgatatcttgctcacccaaactccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgacgatccgtggacgttcggtggaggcaccaagctggaaatcaaacgggctgatgcttcggccgctggatccgaacaaaagctgatctcagaagaagacctaaactcacatcaccatcaccatcactaatctaga

SEQ ID NO: 7 - protein, 1602 aa, Artificial
  Plasmid
METLYSTYRLELEPRTHRALAALAALAGLYLELELELEALAALAGLNPRALAMETALAGLNVALGLNLEGLNGLNSERGLYALAGLLEVALARGPRGLYSERSERVALLYSILESERCYSLYSALASERGLYTYRALAPHESERSERTYRTRPMETASNTRPVALLYSGLNARGPRGLYGLNGLYLEGLTRPILEGLYGLNILETRPPRGLYASPGLYASPTHRASNTYRASNGLYLYSPHELYSGLYLYSALATHRLETHRALAASPGLSERSERSERTHRALATYRMETGLNLESERSERLEALASERGLASPSERALAVALTYRPHECYSALAARGARGGLTHRTHRTHRVALGLYARGTYRTYRTYRALAMETASPTYRTRPGLYGLNGLYTHRSERVALTHRVALSERSERALALYSTHRTHRPRLYSLEGLGLGLYGLPHESERGLALAARGVALASPILELELETHRGLNTHRPRALASERLEALAVALSERLEGLYGLNARGALATHRILESERCYSLYSALASERGLNSERVALASPTYRASPGLYASPSERTYRLEASNTRPTYRGLNGLNILEPRGLYGLNPRPRLYSLELEILETYRASPALASERASNLEVALSERGLYILEPRPRARGPHESERGLYSERGLYSERGLYTHRASPPHETHRLEASNILEHISPRVALGLLYSVALASPALAALATHRTYRHISCYSGLNGLNSERTHRGLASPPRTRPTHRPHEGLYGLYGLYTHRLYSLEGLILELYSARGALAASPALAALAALAALAGLYGLYGLYGLYSERGLYGLYGLYGLYSERGLYGLYGLYGLYSERGLYGLYGLYGLYSERGLNVALGLNLEGLNGLNSERGLYALAGLLEALAARGPRGLYALASERVALLYSMETSERCYSLYSALASERGLYTYRTHRPHETHRARGTYRTHRMETHISTRPVALLYSGLNARGPRGLYGLNGLYLEGLTRPILEGLYTYRILEASNPRSERARGGLYTYRTHRASNTYRASNGLNLYSPHELYSASPLYSALATHRLETHRTHRASPLYSSERSERSERTHRALATYRMETGLNLESERSERLETHRSERGLASPSERALAVALTYRTYRCYSALAARGTYRTYRASPASPHISTYRSERLEASPTYRTRPGLYGLNGLYTHRTHRLETHRVALSERSERALALYSTHRTHRPRLYSLEGLYGLYASPILEVALLETHRGLNSERPRALAILEMETSERALASERPRGLYGLLYSVALTHRMETTHRCYSSERALASERSERSERVALSERTYRMETASNTRPTYRGLNGLNLYSSERGLYTHRSERPRLYSARGTRPILETYRASPTHRSERLYSLEALASERGLYVALPRALAHISPHEARGGLYSERGLYSERGLYTHRSERTYRSERLETHRILESERGLYMETGLALAGLASPALAALATHRTYRTYRCYSGLNGLNTRPSERSERASNPRPHETHRPHEGLYSERGLYTHRLYSLEGLILEASNARGALAASPTHRALAPRTHRGLYSERGLGLNLYSLEILESERGLGLASPLEASNSERHISHISHISHISHISHIS

SEQ ID NO: 8 - protein, 20 aa, Artificial
  linker sequence
ARGTHRVALALAALAPRSER

SEQ ID NO: 9 - protein, 23 aa, Artificial
  Amino acid linker peptide
ALAALAALAGLYGLYPRGLYSER

SEQ ID NO: 10 - protein, 26 aa, Artificial
  linker sequence
SERALAALAALAGLYGLYPRGLYSER

SEQ ID NO: 11 - DNA, 6844 bp, Artificial
  plasmid
acccgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgggtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcggtacccgataaaagcggcttcctgacaggaggccgttttgttttgcagcccacctcaacgcaattaatgtgagttagctcactcattaggcaccccaggctttacactttatgcttccggctcgtatgttgtgtggaattgtgagcggataacaatttcacacaggaaacagctatgaccatgattacgaatttctgaagaaggagatatacatatgaaatacctattgcctacggcagccgctggcttgctgctgctggcagctcagccggccatggcggatatcttgctcacccaaactccagcttctttggctgtgtctctagggcagagggccaccatctcctgcaaggccagccaaagtgttgattatgatggtgatagttatttgaactggtaccaacagattccaggacagccacccaaactcctcatctatgatgcatccaatctagtttctgggatcccacccaggtttagtggcagtgggtctgggacagacttcaccctcaacatccatcctgtggagaaggtggatgctgcaacctatcactgtcagcaaagtactgaggatccgtggacgttcggtggaggcaccaagctggaaatcaaacgtactgttgctgcaccgtctcaggtgcaactgcagcagtctggggctgagctggtgaggcctgggtcctcagtgaagatttcctgcaaggcttctggctatgcattcagtagctactggatgaactgggtgaagcagaggcctggacagggtcttgagtggattggacagatttggcctggagatggtgatactaactacaatggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggtcaaggaacctcagtcaccgtctcctcagccaaaacaacaccccaggtgcagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattacagccttgactactggggccaaggcaccactctcacagtctcctcagccaaaacaacacccaagcttggcggtgatatcgtgctcactcagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgggctgatactgcggccgctggatcccatcaccatcaccatcactaatctagaggcctgtgctaacttaagaaggagatatacatatgaaaaagtggttattagctgcaggtctcggtttagcactggcaacttctgctcaggcggctgacaaaattgcaatcgtcaacatgggcagcctgttccagcaggtagcgcagaaaaccggtgtttctaacacgctggaaaatgagttcaaaggccgtgccagcgaactgcagcgtatggaaaccgatctgcaggctaaaatgaaaaagctgcagtccatgaaagcgggcagcgatcgcactaagctggaaaaagacgtgatggctcagcgccagacttttgctcagaaagcgcaggcttttgagcaggatcgcgcacgtcgttccaacgaagaacgcggcaaactggttactcgtatccagactgctgtgaaacccgttgccaacagccaggatatcgatctggttgttgatgcaaacgccgttgcttacaacagcagcgatgtaaaagacatcactgtcgacgtactgaaacaggttaaataatgctcgaggaactgctgaaacatctgaaggagctgcttaaaggtgagttctgataagcttgacctgtgaagtgaaaaatggcgcacattgtgcgacattttttttgtctgccgtttaccgctactgcgtcacggatccggccgaacaaactccgggaggcagcgtgatgcggcaacaatcacacggatttcccgtgaacggtctgaatgagcggattattttcagggaaagtgagtgtggtcagcgtgcaggtatatgggctatgatgtgcccggcgcttgaggctttctgcctcatgacgtgaaggtggtttgttgccgtgttgtgtggcagaaagaagatagccccgtagtaagttaattttcattaaccaccacgaggcatccctatgtctagtccacatcaggatagcctcttaccgcgctttgcgcaaggagaagaaggccatgaaactaccacgaagttcccttgtctggtgtgtgttgatcgtgtgtctcacactgttgatattcacttatctgacacgaaaatcgctgtgcgagattcgttacagagacggacacagggaggtggcggctttcatggcttacgaatccggtaagtagcaacctagaggcgggcgcaggcccgccttttcaggactgatgctggtctgactactgaagcgcctttataaaggggctgctggttcgccggtagcccctttctccttgctgatgttgtgggaatttcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcgaattccccggggaattcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccctattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 12 - protein, 1422 aa, Artificial
  plasmid
ASPILELELETHRGLNTHRPRALASERLEALAVALSERLEGLYGLNARGALATHRILESERCYSLYSALASERGLNSERVALASPTYRASPGLYASPSERTYRLEASNTRPTYRGLNGLNILEPRGLYGLNPRPRLYSLELEILETYRASPALASERASNLEVALSERGLYILEPRPRARGPHESERGLYSERGLYSERGLYTHRASPPHETHRLEASNILEHISPRVALGLLYSVALASPALAALATHRTYRHISCYSGLNGLNSERTHRGLASPPRTRPTHRPHEGLYGLYGLYTHRLYSLEGLILELYSARGTHRVALALAALAPRSERGLNVALGLNLEGLNGLNSERGLYALAGLLEVALARGPRGLYSERSERVALLYSILESERCYSLYSALASERGLYTYRALAPHESERSERTYRTRPMETASNTRPVALLYSGLNARGPRGLYGLNGLYLEGLTRPILEGLYGLNILETRPPRGLYASPGLYASPTHRASNTYRASNGLYLYSPHELYSGLYLYSALATHRLETHRALAASPGLSERSERSERTHRALATYRMETGLNLESERSERLEALASERGLASPSERALAVALTYRPHECYSALAARGARGGLTHRTHRTHRVALGLYARGTYRTYRTYRALAMETASPTYRTRPGLYGLNGLYTHRSERVALTHRVALSERSERALALYSTHRTHRPRGLNVALGLNLEGLNGLNSERGLYALAGLLEALAARGPRGLYALASERVALLYSMETSERCYSLYSALASERGLYTYRTHRPHETHRARGTYRTHRMETHISTRPVALLYSGLNARGPRGLYGLNGLYLEGLTRPILEGLYTYRILEASNPRSERARGGLYTYRTHRASNTYRASNGLNLYSPHELYSASPLYSALATHRLETHRTHRASPLYSSERSERSERTHRALATYRMETGLNLESERSERLETHRSERGLASPSERALAVALTYRTYRCYSALAARGTYRTYRASPASPHISTYRSERLEASPTYRTRPGLYGLNGLYTHRTHRLETHRVALSERSERALALYSTHRTHRPRLYSLEGLYGLYASPILEVALLETHRGLNSERPRALAILEMETSERALASERPRGLYGLLYSVALTHRMETTHRCYSSERALASERSERSERVALSERTYRMETASNTRPTYRGLNGLNLYSSERGLYTHRSERPRLYSARGTRPILETYRASPTHRSERLYSLEALASERGLYVALPRALAHISPHEARGGLYSERGLYSERGLYTHRSERTYRSERLETHRILESERGLYMETGLALAGLASPALAALATHRTYRTYRCYSGLNGLNTRPSERSERASNPRPHETHRPHEGLYSERGLYTHRLYSLEGLILEASNARGALAASPTHRALAALAALAGLYSERHISHISHISHISHISHIS

SEQ ID NO: 13 - protein, 17 aa, Artificial
  linker sequence
SERALALYSTHRTHRPR

SEQ ID NO: 14 - DNA, 32 bp, Artificial
  primer
gatatacatatgaaatacctattgcctacggc

SEQ ID NO: 15 - DNA, 47 bp, Artificial
  primer
cgaattcttaagttagcacaggcctctagagacacacagatctttag

SEQ ID NO: 16 - DNA, 28 bp, Artificial
  primer
caccctggcgcccaatacgcaaaccgcc

SEQ ID NO: 17 - DNA, 46 bp, Artificial
  primer
ggtatttcatatgtatatctccttcttcagaaattcgtaatcatgg

SEQ ID NO: 18 - DNA, 52 bp, Artificial
  primer
cgaattcttaagaaggagatatacatatgaaaaagtggttattagctgcagg

SEQ ID NO: 19 - DNA, 40 bp, Artificial
  primer
cgaattctcgagcattatttaacctgtttcagtacgtcgg

SEQ ID NO: 20 - DNA, 41 bp, Artificial
  primer
cagccggccatggcggatatcttgctcacccaaactccagc

SEQ ID NO: 21 - DNA, 34 bp, Artificial
  primer
agacggtgcagcaacagtacgtttgatttccagc

SEQ ID NO: 22 - DNA, 41 bp, Artificial
  primer
cgtactgttgctgcaccgtctcaggtgcaactgcagcagtc

SEQ ID NO: 23 - DNA, 45 bp, Artificial
  primer
gaagatggatccagcggccgctgaggagacggtgactgaggttcc

SEQ ID NO: 24 - DNA, 32 bp, Artificial; identical to SEQ ID NO: 14
  primer

SEQ ID NO: 25 - DNA, 47 bp, Artificial; identical to SEQ ID NO: 15
  primer

SEQ ID NO: 26 - DNA, 35 bp, Artificial
  primer
caggcctctagattagtgatggtgatggtgatggg

SEQ ID NO: 27 - DNA, 35 bp, Artificial
  primer
ccggccatggcgcaggtgcagctgcagcagtctgg

SEQ ID NO: 28 - DNA, 21 bp, Artificial
  primer
gctgcccatgttgacgattgc

SEQ ID NO: 29 - DNA, 33 bp, Artificial
  primer
cagccggccatggcgcaggtgcaactgcagcag

SEQ ID NO: 30 - DNA, 36 bp, Artificial
  primer
gaagatggatccagcggccgcagtatcagcccggtt

SEQ ID NO: 31 - DNA, 42 bp, Artificial
  primer
tcacacagaattcttagatctattaaagaggagaaattaacc

SEQ ID NO: 32 - DNA, 40 bp, Artificial
  primer
agcacacgatatcaccgccaagcttgggtgttgttttggc

SEQ ID NO: 33 - DNA, 40 bp, Artificial
  primer
ctgctgcagctgcacctggggtgttgttttggctgaggag

(19) United States
(12) Patent Application Publication
(10) Pub. No.: US 2005/0079170 A1
(43) Pub. Date: Apr. 14, 2005

(54) Dimeric and multimeric antigen binding structure

(76) Inventors: Fabrice Le Gall, Edingen-Neckarhausen (DE); Sergey Kipriyanov, Heidelberg (DE); Uwe Reusch, Maikammer (DE); Gerhard Moldenhauer, Heidelberg (DE); Melvyn Little, Neckargemund (DE)

Correspondence Address: HOWREY SIMON ARNOLD & WHITE, LLP, c/o IP DOCKETING DEPARTMENT, 2941 FAIRVIEW PARK DRIVE, SUITE 200, FALLS CHURCH, VA, 22042-2924, US

(21) Appl. No.: 10/489,626

(22) PCT Filed: Sep. 13, 2002
(86) PCT No.: PCT/EP02/10307

(30) Foreign Application Priority Data
Sep. 14, 2001 (EP) 01122104.1

(51) Int. Cl. A61K 39/395; C07K 16/44

(52) U.S. Cl. 424/132.1; 530/387.3

(57) ABSTRACT

The present invention relates to dimeric and multimeric antigen binding structures, expression vectors encoding said structures, and diagnostic, as well as therapeutic, uses of said structures. The antigen binding structures are preferably in the form of a Fv-antibody construct.

This application is a National Stage of International Application PCT/EP02/10307, filed Sep. 13, 2002, published Mar. 27, 2003 under PCT Article 21(2) in English; which claims the priority of EP 01122104.1 filed Sep. 14, 2001.

### FIELD OF THE INVENTION

The present invention relates to dimeric and multimeric antigen binding structures, expression vectors encoding said structures, and diagnostic as well as therapeutic uses of said structures. The antigen binding structures are preferably in the form of a Fv-antibody construct.

Natural antibodies are themselves dimers, and thus, bivalent. If two hybridoma cells producing different antibodies are artificially fused, some of the antibodies produced by the hybrid hybridoma are composed of two monomers with different specificities. Such bispecific antibodies can also be produced by chemically conjugating two antibodies. Natural antibodies and their bispecific derivatives are relatively large and expensive to produce. The constant domains of mouse antibodies are also a major cause of the human anti-mouse antibody (HAMA) response, which prevents their extensive use as therapeutic agents. They can also give rise to unwanted effects due to their binding of Fc-receptors. For these reasons, molecular immunologists have been concentrating on the production of the much smaller Fab- and Fv-fragments in microorganisms. These smaller fragments are not only much easier to produce, they are also less immunogenic, have no effector functions, and, because of their relatively small size, they are better able to penetrate tissues and tumors. In the case of the Fab-fragments, the constant domains adjacent to the variable domains play a major role in stabilizing the heavy and light chain dimer.

The Fv-fragment is much less stable, and a peptide linker was therefore introduced between the heavy and light chain variable domains to increase stability. This construct is known as a single chain Fv(scFv)-fragment. A disulfide bond is sometimes introduced between the two domains for extra stability. Thus far, tetravalent scFv-based antibodies have been produced by fusion to extra polymerizing domains such as the streptavidin monomer that forms tetramers, and to amphipathic alpha helices. However, these extra domains can increase the immunogenicity of the tetravalent molecule.

Bivalent and bispecific antibodies can be constructed using only antibody variable domains. A fairly efficient and relatively simple method is to make the linker sequence between the V_{H} and V_{L} domains so short that they cannot fold over and bind one another. Reduction of the linker length to 3-12 residues prevents the monomeric configuration of the scFv molecule and favors intermolecular V_{H}-V_{L} pairings with formation of a 60 kDa non-covalent scFv dimer “diabody” (Holliger et al., 1993, Proc. Natl. Acad. Sci. USA 90, 6444-6448). The diabody format can also be used for generation of recombinant bispecific antibodies, which are obtained by the noncovalent association of two single-chain fusion products, consisting of the V_{H} domain from one antibody connected by a short linker to the V_{L} domain of another antibody. Reducing the linker length still further below three residues can result in the formation of trimers (“triabody”, ˜90 kDa) or tetramers (“tetrabody”, ˜120 kDa) (Le Gall et al., 1999, FEBS Letters 453, 164-168). However, the small size of bispecific diabodies (50-60 kDa) leads to their rapid clearance from the blood stream through the kidneys, thus requiring the application of relatively high doses for therapy. Moreover, bispecific diabodies have only one binding domain for each specificity. However, bivalent binding is an important means of increasing the functional affinity, and possibly the selectivity, for particular cell types carrying densely clustered antigens.

Thus, the technical problem underlying the present invention is to provide new dimeric and multimeric antigen binding structures which overcome the disadvantages of the Fv-antibodies of the prior art, and to provide a general way to form a structure with at least four binding domains which is monospecific or multispecific.

The solution to said technical problem is achieved by providing the embodiments characterized in the claims.

The present invention is further described with regard to the figures.

### BRIEF DESCRIPTION OF THE DRAWINGS

FIGS. 1, 2, and 3 depict schemes of the multimeric Fv molecules depending on the particular antibody domains and the length of the peptide linker LL between the variable domains that comprise the multimerization motif.

Abbreviations L0: The V_{H} and V_{L} domains are directly connected without intervening linker peptide; L1: linker sequence coding for Ser residue; L10: linker sequence coding for SerAlaLysThrThrProLysLeuGlyGly polypeptide (SEQ ID NO:1) connecting V_{H} and V_{L} domains; LL: linker sequence coding for (Gly₄Ser)₄ polypeptide (SEQ ID NO:2) connecting hybrid scFv fragments; L18: linker sequence coding for SerAlaLysThrThrProLysLeuGluGluGlyGluPheSerGluAlaArgVal polypeptide (SEQ ID NO:3) connecting V_{H} and V_{L} domains.

FIGS. 4 and 5 depict schemes of construction of the plasmids pHOG scFv₁₈αCD3-LL-scFv₁₀αCD19 and pHOG scFv₁₈αCD19-LL-scFv₁₀αCD3.

Abbreviations c-myc: sequence coding for an epitope recognized by mAb 9E10; His: sequence coding for six C-terminal histidine residues; PelB: signal peptide sequence of the bacterial pectate lyase (PelB leader); rbs: ribosome binding site; Stop: stop codon (TAA); V_{H} and V_{L}: variable region of the heavy and light chain; L10: linker sequence coding for SerAlaLysThrThrProLysLeuGlyGly polypeptide connecting V_{H} and V_{L} domains; LL: linker sequence coding for (Gly₄Ser)₄ polypeptide connecting hybrid scFv-fragments; L18: linker sequence coding for SerAlaLysThrThrProLysLeuGluGluGlyGluPheSerGluAlaArgVal polypeptide connecting V_{H} and V_{L} domains; B: BamHI, Ea: EagI, E: EcoRI, Nc: NcoI; N. NotI, P: PvuII, X: XbaI.

FIG. 6 shows nucleotide (SEQ ID NO:4) and deduced amino acid sequences (SEQ ID NO:5) of the plasmid pSKK2 scFv3−LL-Db19.

Abbreviations His6 tail: sequence coding for six C-terminal histidine residues; β-lactamase: gene encoding β-lactamase that confers resistance to ampicillin resistance; bp: base pairs; c-myc epitope: sequence coding for an epitope recognized by mAb 9E10; Lac P/0: wt lac operon promoter/operator; Pel B leader: signal peptide sequence of the bacterial pectate lyase; V_{H} and H_{L}: variable region of the heavy and light chain; L10: linker sequence coding for SerAlaLysThrThrProLysLeuGlyGly polypeptide connecting V_{H} and V_{L} domains; LL: linker sequence coding for (Gly₄Ser)₄ polypeptide connecting hybrid scFv-fragments; LI8: linker sequence coding for SerAlaLysThrThrProLysLeuGluGluGlyGluPheSerGluAlaArgVal polypeptide connecting V_{H} and V_{L} domains; rbs: ribosome binding site; V_{H} and V_{L}: variable region of the heavy and light chain; hok-sok: plasmid stabilizing DNA locus; lacI: gene coding for lac-repressor; lac P/0: wt lac operon promoter/operator; lacZ′: gene coding for α-peptide of β-galactosidase; skp gene: gene encoding bacterial periplasmic factor Skp/OmpH; tLPP: nucleotide sequence of the lipoprotein terminator; M13 ori: origin of the DNA replication; pBR322ori: origin of the DNA replication; tHP: strong terminator of transcription; SD1: ribosome binding site (Shine Dalgarno) derived from E. coli lacZ gene (lacZ); SD2 and SD3: Shine Dalgarno sequence for the strongly expressed T7 gene 10 protein (T7g10).

FIG. 7 shows nucleotide (SEQ ID NO:6; FIG. 7a) and deduced amino acid (SEQ ID NO:7; FIG. 7b) sequences of the plasmid pSKK2 scFv19-LL-Db3.

Abbreviations His6 tail: sequence coding for six C-terminal histidine residues; β-lactamase: gene encoding β-lactamase that confers resistance to ampicillin resistance; bp: base pairs; c-myc epitope: sequence coding for an epitope recognized by mAb 9E10; Lac P/0: wt lac operon promoter/operator; PelB leader: signal peptide sequence of the bacterial pectate lyase; V_{H} and V_{L}: variable region of the heavy and light chain; L10: linker sequence coding for SerAlaLysThrThrProLysLeuGlyGly polypeptide connecting V_{H} and V_{L} domains; LL: linker sequence coding for (Gly4Ser)4 polypeptide connecting hybrid scFv-fragments; Ll8: linker sequence encoding SerAlaLysThrThrProLysLeuGluGluGlyGluPheSerGluAlaArgVal polypeptide connecting V_{H} and V_{L} domains; rbs: ribosome binding site; hok-sok: plasmid stabilizing DNA locus; lacI: gene coding for lac-repressor; lac P/0: wt lac operon promoter/operator; lacZ′: gene coding for α-peptide of β-galactosidase; skp gene: gene encoding bacterial periplasmic factor Skp/OmpH; tLPP: nucleotide sequence of the lipoprotein terminator; M13 ori: origin of the DNA replication, pBR322ori: origin of the DNA replication; tHP: strong terminator of transcription; SD1: ribosome binding site (Shine Dalgarno) derived from E. coli lacZ gene (lacZ); SD2 and SD3: Shine Dalgarno sequence for the strongly expressed T7 gene 10 protein (T7g10).

FIG. 8 shows analyses of protein contents of peaks after IMAC.

Electrophoresis was carried out under reducing conditions; Western blot with anti-c-myc monoclonal antibody, in the case of scFv3−Db19 (FIG. 8A) and scFv 19×Db3 (FIG. 8B) molecules.

FIG. 9 shows size-exclusion FPLC chromatography elution profiles.

A calibrated Superdex 200 HR10/30 column was used and the analysis of protein contents of peaks by Western blot was carried out with anti-c-myc monoclonal antibody, in the case of scFv3−Db19 (FIG. 9A) and scFv19−Db3 (FIG. 9B) molecules.

FIG. 10 shows size-exclusion FPLC chromatography elution profiles. A calibrated Superdex 200 HR10/30 column for the scFv3−Db19, scFv19−Db3, scFv19−scFv3 and scFv3−scFv19 molecules was used.

FIG. 11 shows flow cytometry results on CD3⁺Jurkat and CD19⁺JOK-1 cells.

FIG. 12 shows an analysis of cell surface retention on CD19⁺JOK-1 (A,B) and CD3⁺Jurkat cells (C,D) for the scFv3−scFv19 and scFv3−Db19 molecules (A,C) and for scFv19−scFv3 and scFv 19×Db3 molecules (B,D).

FIG. 13 shows depletion of primary malignant CD19⁺ CLL-cells by recruitment of autologous T-lymphocytes through CD19×CD3 bispecific molecules.

Freshly isolated peripheral blood mononuclear cells (PBMC) from patient with chronic lymphocytic leukemia (CLL) were seeded in individual wells of a 12-well plate in 2 ml RPMI-Medium/10% FCS at a density of 2×10⁶ cells/ml. The recombinant antibodies scFv3−scFv19 and scFv3−Db19 were added at concentration of 5 μg/ml. After 5 day incubation, the cells were harvested, counted, and stained with anti-CD3 MAb OKT3, anti-CD4 MAb Edu-2, anti-CD8 MAb UCH-T4, and anti-CD19 MAb HD37 for flow cytometric analysis. 10⁴ living cells were analyzed using a Beckman-Coulter flow cytometer and the relative amounts of CD3⁺, CD4⁺, CD8⁺ and CD19⁺ cells were plotted.

FIG. 14 is a schematic representation of the multimeric Fv-antibody construct formed by dimerizing via N-terminal “diabody” motif.

Abbreviations L7: 7 amino acid linker peptide Arg-Thr-Val-Ala-Ala-Pro-Ser (SEQ 10 NO:8) connecting the V_{L} and V_{H} domains in the dimerizing “diabody” motif; SL: 8 amino acid linker peptide Ala-Ala-Ala-Gly-Gly-Pro-Gly-Ser (SEQ ID NO:9) between the dimerizing motif and scFvs; L18: 18 amino acid linker peptide Ser-Ala-Lys-Thr-Thr-Pro-Lys-Leu-Glu-Glu-Gly-Glu-Phe-Ser-Glu-Ala-Arg-Val connecting the V_{H} and V_{L} domains in scFvs.

FIG. 15 is a diagram of the expression plasmid pSKK3-scFv_{L7}anti-CD19-SL-scFv_{L18}anti-CD3.

Abbreviations bla: gene of beta-lactamase responsible for ampicillin resistance; bp: base pairs; CDR-H1, CDR-H2 and CDR-H3: sequence encoding the complementarity determining regions (CDR) 1-3 of the heavy chain; CDR-L1, CDR-L2 and CDR-L3: sequence encoding the complementarity determining regions (CDR) 1-3 of the light chain; His6 tag: sequence encoding six C-terminal histidine residues; hok-sok: plasmid stabilizing DNA locus; L7 linker: sequence which encodes the 7 amino acid peptide Arg-Thr-Val-Ala-Ala-Pro-Ser connecting the anti-CD19 V_{L} and V_{H} domains; L18 linker: sequence which encodes the 18 amino acid peptide Ser-Ala-Lys-Thr-Thr-Pro-Lys-Leu-Glu-Glu-Gly-Glu-Phe-Ser-Glu-Ala-Arg-Val connecting the anti-CD3 V_{H} and V_{L} domains; lacI: gene encoding lac-repressor; lac P/O: wild-type lac-operon promoter/operator; M13ori: intergenic region of bacteriophage M13; pBR322ori: origin of the DNA replication; PelB leader: signal peptide sequence of the bacterial pectate lyase; SD1: ribosome binding site derived from E. coli lacZ gene (lacZ); SD2 and SD3: ribosome binding site derived from the strongly expressed gene 10 of bacteriophage T7 (T7g10); skp gene: gene encoding bacterial periplasmic factor Skp/OmpH; SL linker: sequence which encodes the 9 amino acid peptide Ser-Ala-Ala-Ala-Gly-Gly-Pro-Gly-Ser (SEQ 10 No:10) connecting the anti-CD19 and anti-CD3 V_{H} domains; tHP: strong transcriptional terminator; tLPP: lipoprotein terminator of transcription; V_{H} and V_{L}: sequence coding for the variable region of the immunoglobulin heavy and light chain, respectively. Unique restriction sites are indicated.

FIG. 16 shows nucleotide (FIG. 16a) and deduced amino acid (FIG. 16b) sequences of the plasmid pSKK3-scFv_{L7}anti-CD19-SL-scFv_{L18}anti-CD3.

FIG. 17 shows an analysis of purified Db19-SL-scFv3 molecule by 12% sodium dodecyl sulfate-polyacrylamide gel electrophoresis (SDS-PAGE) under reducing conditions.

Lane 1: Mᵣ markers (kDa, Mᵣ in thousands) Lane 2: Db19-SL-scFv3. The gel was stained with Coomassie Blue.

FIG. 18 shows an analysis of purified Db19-SL-scFv3 molecule by size exclusion chromatography on a calibrated Superdex 200 column.

The elution positions of molecular mass standards are indicated.

FIG. 19 shows a Lineweaver-Burk analysis of fluorescence dependence on antibody concentration as determined by flow cytometry.

Binding of Db19-SL-scFv3 to CD3⁺ Jurkat (A) and CD19⁺ JOK-1 cells (B) was measured.

FIG. 20 shows depletion of primary malignant CD19⁺ CLL-cells by recruitment of autologous T-lymphocytes through Db19-SL-scFv3 molecule.

Freshly isolated peripheral blood mononuclear cells (PBMC) from a patient with chronic lymphocytic leukemia (CLL) were seeded in individual wells of a 12-well plate in 2 ml RPMI-Medium/10% FCS at a density of 2×10⁶ cells/ml. The recombinant scFv-antibody Db19-SL-scFv3 or CD19×CD3 tandem diabody (Tandab; Kipriyanov et al. 1999, J. Mol. Biol. 293, 41-56; Cochlovius et al. 2000, Cancer Res. 60, 4336-4341) was added at concentrations of 5 μg/ml, 1 μg/ml, and 0.1 μg/ml. After 5 day incubation, the cells were harvested, counted, and stained with anti-CD3 MAb OKT3, anti-CD4 MAb Edu-2, anti-CD8 MAb UCH-T4, and anti-CD19 MAb HD37 for flow cytometric analysis. 10⁴ living cells were analyzed using a Beckman-Coulter flow cytometer and the relative amounts of CD3⁺, CD4⁺, CD8⁺ and CD19⁺ cells were plotted. n.d.: not determined due to CD19 coating and/or modulation.

FIG. 21 is a schematic representation of the multimeric scFv₇-L₆-scFv₁₀ Fv-antibody construct formed by dimerizing via N-terminal “diabody” motif.

Abbreviations L7: 7 amino acid linker peptide Arg-Thr-Val-Ala-Ala-Pro-Ser connecting the V_{L} and V_{H} domains in the dimerizing “diabody” motif; L6: 6 amino acid linker peptide Ser-Ala-Lys-Thr-Thr-Pro (SEQ ID NO:13) between the dimerizing motif and scFvs; L10: 10 amino acid linker peptide Ser-Ala-Lys-Thr-Thr-Pro-Lys-Leu-Gly-Gly connecting the V_{H} and V_{L} domains in the scFvs.

FIG. 22 is a diagram of the expression plasmid pSKK3-scFv_{L7}anti-CD19-L6-scFv_{L10}anti-CD3.

Abbreviations bla: gene of beta-lactamase responsible for ampicillin resistance; bp: base pairs; CDR-H1, CDR-H2 and CDR-H3: sequence encoding the complementarity determining regions (CDR) 1-3 of the heavy chain; CDR-L1, CDR-L2 and CDR-L3: sequence encoding the complementarity determining regions (CDR) 1-3 of the light chain; His6 tag: sequence encoding six C-terminal histidine residues; hok-sok: plasmid stabilizing DNA locus; L6 linker: sequence which encodes the 6 amino acid peptide Ser-Ala-Lys-Thr-Thr-Pro connecting the anti-CD19 and anti-CD3 V_{H} domains; L7 linker: sequence which encodes the 7 amino acid peptide Arg-Thr-Val-Ala-Ala-Pro-Ser connecting the anti-CD19 V_{L} and V_{H} domains; L10 linker: sequence which encodes the 10 amino acid peptide Ser-Ala-Lys-Thr-Thr-Pro-Lys-Leu-Gly-Gly connecting the anti-CD3 V_{H} and V_{L} domains; lacI: gene encoding lac-repressor; lac P/O: wild-type lac-operon promoter/operator; M13ori: intergenic region of bacteriophage M13; pBR322ori: origin of the DNA replication; PelB leader: signal peptide sequence of the bacterial pectate lyase; SD1: ribosome binding site derived from E. coli lacZ gene (lacZ); SD2 and SD3: ribosome binding site derived from the strongly expressed gene 10 of bacteriophage T7 (T7g10); skp gene: gene encoding bacterial periplasmic factor Skp/OmpH; tHP: strong transcriptional terminator; tLPP: lipoprotein terminator of transcription; V_{H} and V_{L}: sequence coding for the variable region of the immunoglobulin heavy and light chain, respectively. Unique restriction sites are indicated.

FIG. 23 shows nucleotide (SEQ ID NO:11; FIG. 23a) and deduced amino acid (SEQ ID NO:1; FIG. 23b) sequences of the plasmid pSKK3-scFv_{L7}anti-CD19-L6-scFv_{L10}anti-CD3

FIG. 24 shows an analysis of purified Db19-L6-scFv3 molecule by 12% sodium dodecyl sulfate-polyacrylamide gel electrophoresis (SDS-PAGE) under reducing conditions.

Lane 1: Mᵣ markers (kDa, Mᵣ in thousands) Lane 2: Db19-L6-scFv3. The gel was stained with Coomassie Blue.

FIG. 25 shows an analysis of purified Db19-L6-scFv3 molecule by size exclusion chromatography on a calibrated Superdex 200 column.

The elution positions of molecular mass standards are indicated.

FIG. 26 shows a Lineweaver-Burk analysis of fluorescence dependence on concentration of Db19-L6-scFv3 as determined by flow cytometry.

Binding of Db19-L6-scFv3 to CD3⁺ Jurkat (A) and CD19⁺JOK-1 cells (B) was measured.

### DETAILED DESCRIPTION OF THE INVENTION

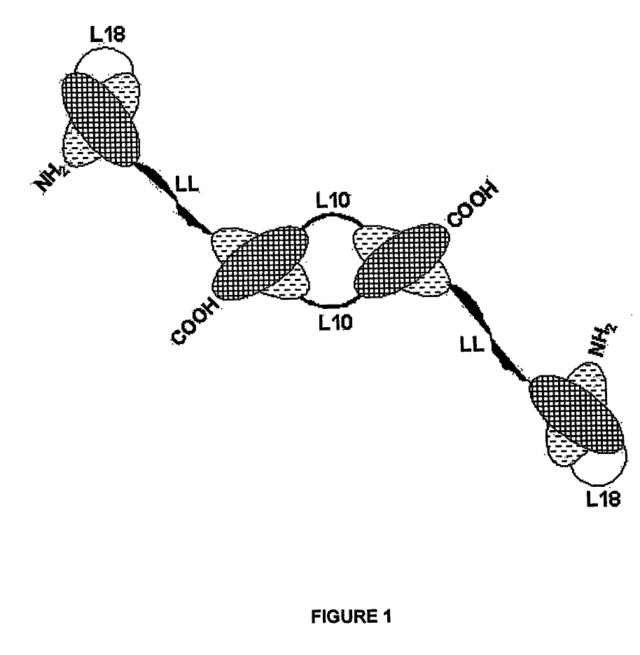

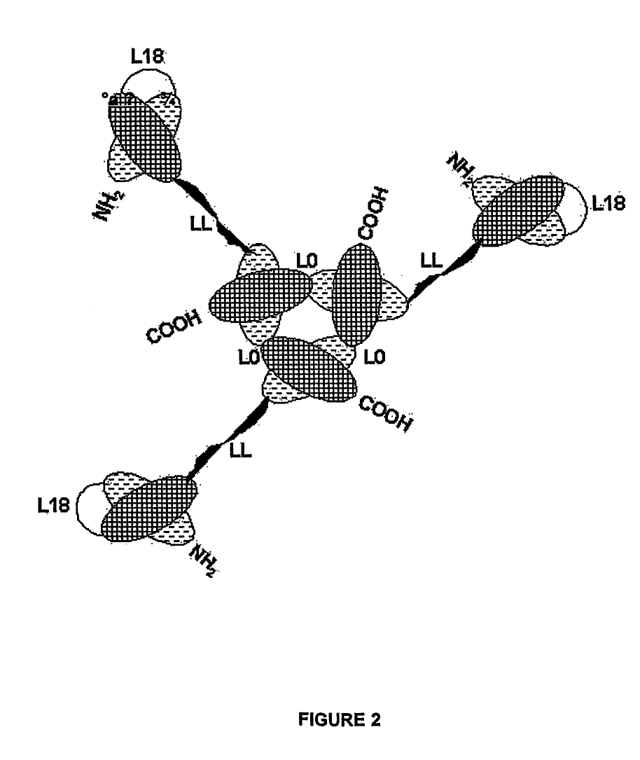

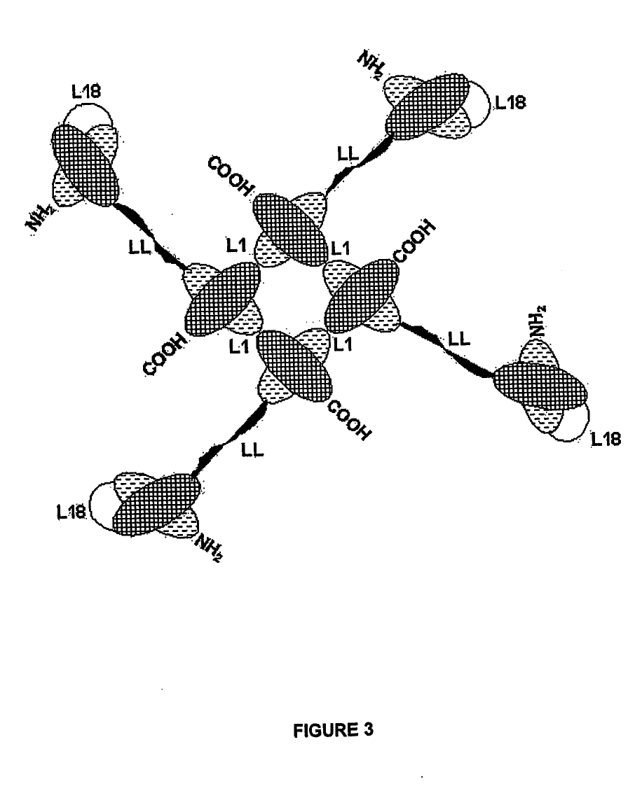

The present invention is based on the observation that scFv-dimers, -trimers and -tetramers that are placed in the N-terminal or C-terminal part of the molecule can be used as multimerization motifs for construction of multimeric Fv-molecules. Thus, the present invention provides a general way to form a multimeric Fv molecule with at least four binding domains which is monospecific or multispecific. Each monomer of the Fv molecule of the present invention is characterized by a V_{H}/V_{L} antigen-binding unit and two antibody variable domains that form V_{H}/V_{L} antigen-binding units after binding non-covalently to the variable domains of other monomers (multimerization motif). Dimers, trimers or tetramers are formed depending on the variable domains and the length of the peptide linkers between the variable domains that comprise the multimerization motif (see FIGS. 1, 2, 3, 14, 21).

The dimeric or multimeric antigen binding structures of the present invention, preferably in form of multimeric Fv-antibodies, are expected to be very stable and have a higher binding capacity. They should also be particularly useful for therapeutic purposes, since the dimeric diabodies used so far are small and remove fairly quickly from the blood stream through the kidneys. Moreover, the single chain format of the multimeric Fv-antibodies of the present invention allows them to be made in eukaryotic organisms and not only in bacteria.

Accordingly, the present invention relates to a dimeric or multimeric structure comprising a single chain molecule that comprises four antibody variable domains, wherein
(a) either the first two or the last two of the four variable domains bind intramolecularly to one another within the same chain by forming an antigen binding scFv in the orientation V_{H}/V_{L} or V_{L}/V_{H}
(b) the other two domains bind intermolecularly with the corresponding V_{H} or V_{L} domains of another chain to form antigen binding V_{H}/V_{L} pairs.

In a particularly preferred embodiment the present invention relates to a multimeric Fv-antibody, characterized by the following features:
(a) the monomers of said Fv-antibody comprise at least four variable domains of which two neighboring domains of one monomer form an antigen-binding V_{H}-V_{L} or V_{L}-V_{H} scFv unit; these two variable domains are linked by a peptide linker of at least 5 amino acid residues, preferably of at least 6, 7, 8, 9, 10, 11, or 12 amino acids, which does not prevent the intramolecular formation of a scFv,
(b) at least two variable domains of the monomer are non-covalently bound to two variable domains of another monomer resulting in the formation of at least two additional antigen binding sites to form the multimerization motif; these two variable domains of each monomer are linked by a peptide linker of a maximum of 12, preferably a maximum of 10 amino acid residues.

A further preferred feature is that the antigen-binding V_{H}-V_{L} or V_{L}-V_{H} scFv unit formed by the two neighbouring domains of one monomer is linked to the other variable domains of the multimerization motif by a peptide linker of at least 5 amino acid residues, preferably of at least 6, 7, 8, 9, 10, 11, or 12 amino acid residues.

The term “Fv-antibody” relates to an antibody containing variable domains but not constant domains.

The term “peptide linker” relates to any peptide capable of connecting two variable domains with its length depending on the kinds of variable domains to be connected. The peptide linker might contain any amino acid residue with the amino acid residues glycine, serine and proline being preferred for the peptide linker linking the second and third variable domain.

The term “intramolecularly” means interaction between V_{H} and V_{L} domains which belong to the same polypeptide chain (monomer) with the formation of a functional antigen binding site.

The term “intermolecularly” means interaction of the V_{H} and V_{L} domains which belong to different monomers.

The dimeric or multimeric antigen binding construct, e.g., the multimeric Fv-antibody of the present invention, can be prepared according to standard methods. Preferably, said Fv-antibody is prepared by ligating DNA sequences encoding the peptide linkers with the DNA sequences encoding the variable domains, such that the peptide linkers connect the variable domains, resulting in the formation of a DNA sequence encoding a monomer of the multimeric Fv-antibody and expressing DNA sequences encoding the various monomers in a suitable expression system as described in the Examples below.

The antigen binding structures, in particular the Fv-antibodies, of the present invention can be further modified using conventional techniques known in the art, for example, by using amino acid deletion(s), insertion(s), substitution(s), addition(s), and/or recombination(s), and/or any other modification(s) known in the art either alone or in combination. Methods for introducing such modifications in the DNA sequence underlying the amino acid sequence of a variable domain or peptide linker are well known to the person skilled in the art; see, e.g., Sambrook, Molecular Cloning: A Laboratory Manual, 2^{nd} Edition, Cold Spring Harbor Laboratory (1989) N.Y.

The antigen binding structures of the present invention can comprise at least one further protein domain, said protein domain being linked by covalent or non-covalent bonds. The linkage can be based on genetic fusion according to the methods known in the art and described above, or can be performed by, e.g., chemical cross-linking as described in, e.g., WO 94/04686. The additional domain present in the fusion protein comprising the structure employed in accordance with the invention may be linked preferably by a flexible linker, and advantageously by a peptide linker, wherein said peptide linker comprises plural, hydrophilic, peptide-bonded amino acids of a length sufficient to span the distance between the C-terminal end of said further protein domain and the N-terminal end of the Fv-antibody, or vice versa. The above described fusion protein may further comprise a cleavable linker or cleavage site for proteinases. The fusion protein may also comprise a tag, like a histidine-tag, e.g., (His)₆.

In a preferred embodiment of the present invention, the monomers of the antigen binding structure comprise four variable domains, and either the first and second, or the third and fourth, variable domains of the monomers are linked by a peptide linker of 12, 11, 10, or less amino acid residues, preferably less than five amino acid residues. In an even more preferred embodiment, either the first and second or the third and fourth domain are linked directly without intervening amino acid residues.

In another preferred embodiment of the present invention, the second and third variable domains of the monomers are linked by a peptide linker of at least 5 amino acid residues, preferably of at least 6, 7, 8, 9, 10, 11, or 12. Preferably, the maximum number of amino acid residues is 30.

In another preferred embodiment of the present invention, any variable domain is shortened by at least one amino acid residue at its N- and/or C-terminus. In some circumstances, this truncated form gives a better stability of the molecule, as described in German patent application 100 63 048.0.

In a particularly preferred embodiment of the present invention, the order of domains of a monomer is V_{H}-V_{L}-V_{H}-V_{L},V_{L}-V_{H}-V_{H}-V_{L}, V_{H}-V_{L}-V_{L}-V_{H} or V_{L}-V_{H}-V_{L}-V_{H}.

In some cases, it might be desirable to strengthen the association of two variable domains. Accordingly, in a further preferred embodiment of the multimeric Fv-antibody of the present invention, the binding of at least one pair of variable domains is strengthened by at least one intermolecular disulfide bridge. This can be achieved by modifying the DNA sequences encoding the variable domains accordingly, i.e., by introducing cysteine codons. The two most promising sites for introducing disulfide bridges appeared to be V_{H}44-V_{L}100 connecting framework 2 of the heavy chain with framework 4 of the light chain and V_{H}105-V_{L}43 that links framework 4 of the heavy chain with framework 2 of the light chain.

In a further preferred embodiment of the present invention, the multimeric Fv-antibody is a tetravalent dimer, hexavalent trimer, or octavalent tetramer. The formation of such forms is preferably determined by particular V_{H} and V_{L} domains comprising the multimerization motif and by the length of the linker.

In another preferred embodiment of the present invention, the multimeric Fv-antibody is a bispecific, trispecific, tetraspecific, . . . etc. antibody.

Multimerization of the monomeric subunits can be facilitated by the presence of a dimerization motif at the C-terminus of the fourth variable domain, which is, preferably, a (poly)peptide directly linked via a peptide bond. Examples of such dimerization motifs are known to the person skilled in the art and include streptavidin and amphipathic alpha helixes. Accordingly, in a further preferred embodiment, a dimerization motive is fused to the last domain of at least two monomers of the multimeric Fv-antibody of the present invention.

For particular therapeutic applications, at least one monomer of the multimeric antibody of the invention can be linked non-covalently or covalently to a biologically active substance (e.g., cytokines or growth hormones), a chemical agent (e.g. doxorubicin, cyclosporin), a peptide (e.g., α-Amanitin), a protein (e.g., granzyme A and B).

In an even more preferred embodiment, the multimeric Fv-antibody of the present invention is (I) a monospecific antibody capable of specifically binding the CD19 antigen of B-lymphocytes or the carcinoma embryonic antigen (CEA); or (II) a bispecific antibody capable of specifically binding (a) CD19 and the CD3 complex of the T-cell receptor, (b) CD19 and the CD5 complex of the T-cell receptor, (c) CD19 and the CD28 antigen on T-lymphocytes, (d) CD19 and CD16 on natural killer cells, macrophages and activated monocytes, (e) CEA and CD3, (f) CEA and CD28, or (g) CEA and CD16. The nucleotide sequences of the variable domains have already been obtained and described in the case of the antibody anti-CD19 (Kipriyanov et al., 1996, J. Immunol. Methods 200, 51-62), anti-CD3 (Kipriyanov et al., 1997, Protein Engineer. 10, 445-453), anti-CD28 (Takemura et al.; 2000, FEBS Lett. 476, 266-271), anti-CD16 (German Patent Application DE 199 37 264 A1), anti-CEA (Griffiths et al., 1993, EMBO J. 12, 725-734), and anti-CD5 (Better et al., 1993, Proc. Natl. Acad. Sci. U.S.A. 90, 457-461).

Surprisingly, a tetravalent structure as defined in claim 1 with the specificities anti-CD3 and anti-CD19 showed a much higher efficacy in vitro than a corresponding bivalent (scFv)x2 structure and a tetravalent structure in which all of the four domains formed pairs with corresponding domains of another chain.

Another object of the present invention is a process for the preparation of a multimeric Fv-antibody according to the present invention, wherein (a) DNA sequences encoding the peptid linkers are ligated with the DNA sequences encoding the variable domains, such that the peptide linkers connect the variable domains, resulting in the formation of a DNA sequence encoding a monomer of the multimeric Fv-antibody, and (b) the DNA sequences encoding the various monomers are expressed in a suitable expression system. The various steps of this process can be carried according to standard methods, e.g., methods described in Sambrook et al., or described in the Examples below.

The present invention also relates to DNA sequences encoding the multimeric Fv-antibody of the present invention and vectors, preferably expression vectors containing said DNA sequences.

A variety of expression vector/host systems may be utilized to contain and express sequences encoding the multimeric Fv-antibody. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. The invention is not limited by the host cell employed.

The “control elements” or “regulatory sequences” are those non-translated regions of the vector-enhancers, promoters, 5′ and 3′ untranslated regions-which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the Bluescript.R™ phagemid (Stratagene, LaJolla, Calif.) or pSport1.™ plasmid (Gibco BRL) and the like may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding the multimeric Fv-antibody, vectors based on SV40 or EBV may be used with an appropriate selectable marker.

In bacterial systems, a number of expression vectors may be selected depending upon the use intended for the multimeric Fv-antibody. Vectors suitable for use in the present invention include, but are not limited to, the pSKK expression vector for expression in bacteria.

In the yeast, Saccharomyces cerevisiae, a number of vectors containing constitutive or inducible promoters such as alpha factor, alcohol oxidase, and PGH may be used. For reviews, see Grant et al. (1987) Methods Enzymol. 153:516-544.

In cases where plant expression vectors are used, the expression of sequences encoding the multimeric Fv-antibody may be driven by any of a number of promoters. For example, viral promoters such as the 35S and 19S promoters of CaMV may be used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196.

An insect system may also be used to express the multimeric Fv-antibody. For example, in one such system, Autographa californica nuclear polyhedrosis virus (AcNPV) is used as a vector to express foreign genes in Spodoptera frugiperda cells or in Trichoplusia larvae. The sequences encoding the multimeric Fv-antibody may be cloned into a non-essential region of the virus, such as the polyhedrin gene, and placed under control of the polyhedrin promoter. Successful insertion of the multimeric Fv-antibody will render the polyhedrin gene inactive and produce recombinant virus lacking coat protein. The recombinant viruses may then be used to infect, for example, S. frugiperda cells or Trichoplusia larvae in which APOP may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, sequences encoding the multimeric Fv-antibody may be ligated into an adenovirus transcription/translation complex consisting of the late promoter and tripartite leader sequence. Insertion in a non-essential E1 or E3 region of the viral genome may be used to obtain a viable virus which is capable of expressing the multimeric Fv-antibody in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of 6 to 10M are constructed and delivered via conventional delivery methods (liposomes, polycationic amino polymers, or vesicles) for therapeutic purposes.

Specific initiation signals may also be used to achieve more efficient translation of sequences encoding the multimeric Fv-antibody. Such signals include the ATG initiation codon and adjacent sequences. In cases where sequences encoding the multimeric Fv-antibody, its initiation codon, and upstream sequences are inserted into the appropriate expression vector, no additional transcriptional or translational control signals may be needed. However, in the case where only the coding sequence is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Post-translational processing which cleaves a “prepro” form of the protein may also be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; Bethesda, Md.), and may be chosen to ensure the correct modification and processing of the foreign protein.

For long-term, high-yield production of recombinant proteins, stable expression is preferred. For example, cell lines which stably express the multimeric Fv-antibody may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk.sup.- or aprt.sup.- cells, respectively. Also, antimetabolite, antibiotic, or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

A particular expression vector is pSKK2-scFv_{L18}anti-CD3-LL-scFv_{L10}anti-CD19(pSKK2-scFv3LL Db19) (deposited with the DSMZ according to the Budapest Treaty under DSM 14470 at Aug. 22, 2001 or pSKK2-scFv_{L18}anti-CD19-LL-scFv_{L10}anti-CD3(pSKK2-scFv19LL Db3) (deposited with the DSMZ according to the Budapest Treaty under DSM 14471 at Aug. 22, 2001.

The present invention also relates to a pharmaceutical composition containing a multimeric Fv-antibody of the present invention, a DNA sequence, or an expression vector, preferably combined with suitable pharmaceutical carriers. Examples of suitable pharmaceutical carriers are well known in the art and include phosphate buffered saline solutions, water, emulsions, such as oil/water emuslions, various types of wetting agents, sterile solutions etc. Such carriers can be formulated by conventional methods and can be administered to the subject at a suitable dose. Administration of the suitable compositions may be effected by different ways, e.g., by intravenous, intraperetoneal, subcutaneous, intramuscular, topical, or intradermal administration. The route of administration, of course, depends on the nature of the disease, e.g., tumor, and the kind of compound contained in the pharmaceutical composition. The dosage regimen will be determined by the attending physician and other clinical factors. As is well known in the medical arts, dosages for any one patient depend on many factors, including the patient's size, body surface area, age, sex, the particular compound to be administered, time and route of administration, the kind of the disorder, general health, and other drugs being administered concurrently.

Preferred medical uses of the compounds of the present invention are: (a) the treatment of a viral, bacterial, tumoral, or prion related diseases, (b) the agglutination of red blood cells, (c) linking cytotoxic cells, e.g., T or Natural killer cells of the immune system to tumor cells, or (d) linking activating cytokines, preferably IL-1, IL-2, IFNγ, TNFα, or GM-CSF, cytotoxic substances (e.g., doxorubicin, cyclosporin, α-Amanitin), or a protease, preferably Granzyme B, to a target cell.

A further object of the present invention is the use of a multimeric Fv-antibody of the present invention for diagnosis. For use in the diagnostic research, kits are also provided by the present invention, said kits comprising a multimeric antibody of the present invention. The Fv-antibody can be detectably labeled. In a preferred embodiment, said kit allows diagnosis by ELISA, and contains the Fv-antibody bound to a solid support, for example, a polystyrene microtiter dish or nitrocellulose paper, using techniques known in the art. Alternatively, said kit is based on a RIA, and contains said Fv-antibody marked with a radioactive isotope. In a preferred embodiment of the kit of the invention, the antibody is labelled with enzymes, fluorescent compounds, luminescent compounds, ferromagnetic probes, or radioactive compounds.

The following Examples illustrate the invention.

### EXAMPLES

### Example 1

### Construction of the plasmids pSKK2 scFv_{L18}anti-CD3-LL-scFv_{L10}anti-CD19 (scFv3−Db19) and pSKK2 scFv_{L18}anti-CD19-LL-scFv_{L10}anti-CD3 (scFv19−Db3) for Expression of Multimeric Fv Molecules in Bacteria

For generation of multimeric Fv constructs, the plasmids pHOG_HD37, pHOG_Dia_HD37, pHOG_mOKT3+NotI and pHOG_Dia_mOKT3 encoding the antibody fragments were derived either from hybridoma HD37 specific to human CD19 (Kipriyanov et al., 1996, J. Immunol. Meth. 196, 51-62; Le Gall et al., 1999, FEBS Lett., 453, 164-168) or from hybridoma OKT3 specific to human CD3 (Kipriyanov et al., 1997, Protein Eng. 10, 445-453) were used.

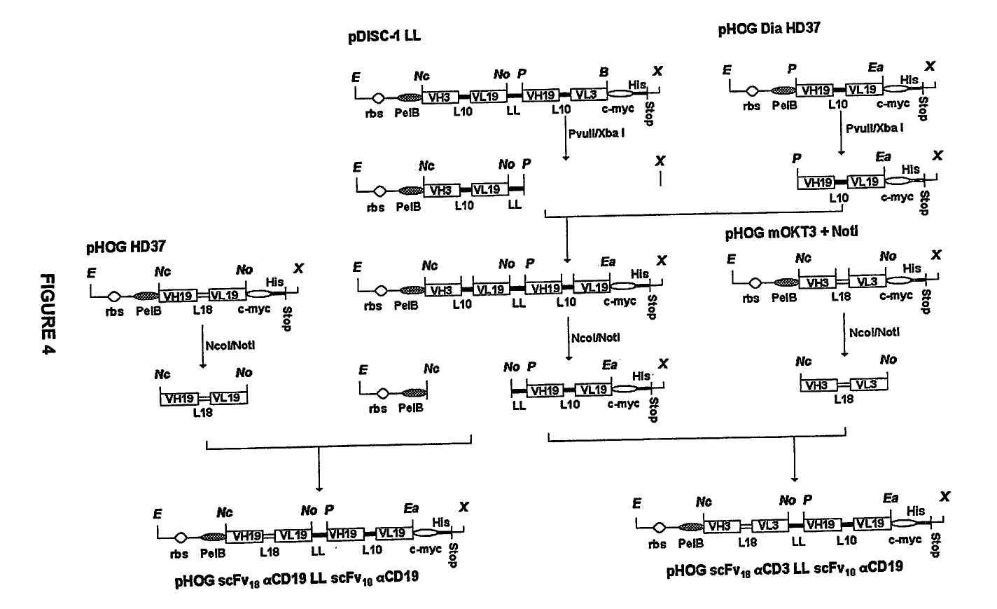

The anti-CD19 ScFV_{L10} gene followed by a segment coding for a c-myc epitope and a hexahistidinyl tail was cut with PvuII/XbaI from the plasmid pHOG Dia HD37, and recloned into the PvuII/XbaI linearized vector pDISC-1 LL (Kipriyanov et al., 1999, J. Mol. Biol. 293, 41-56) (FIG. 4). This hybrid plasmid was linearized by NcoI/NotI and the gene coding for the scFV_{L18} (cut by NcoI/NotI from the plasmid pHOG mOKT3+NotI) was ligated into this plasmid. The plasmid obtained is the pHOG scFv_{L18}αCD3-LL-scFv_{L10}αCD19 (scFv3−Db19) (FIG. 4).

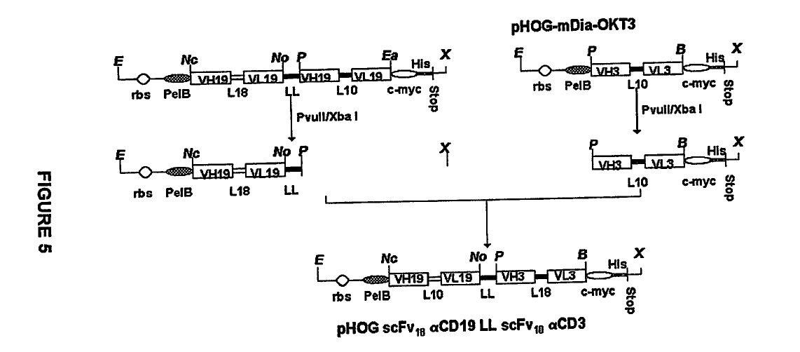

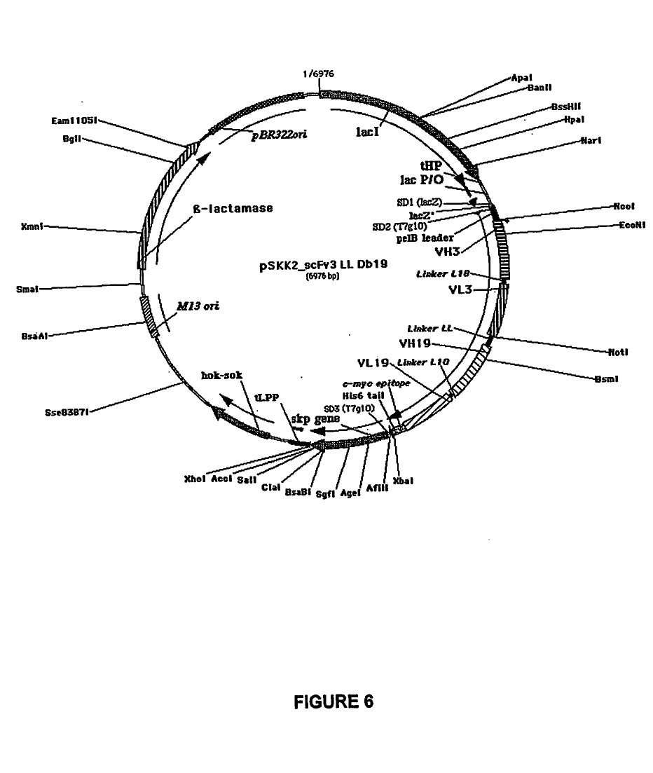

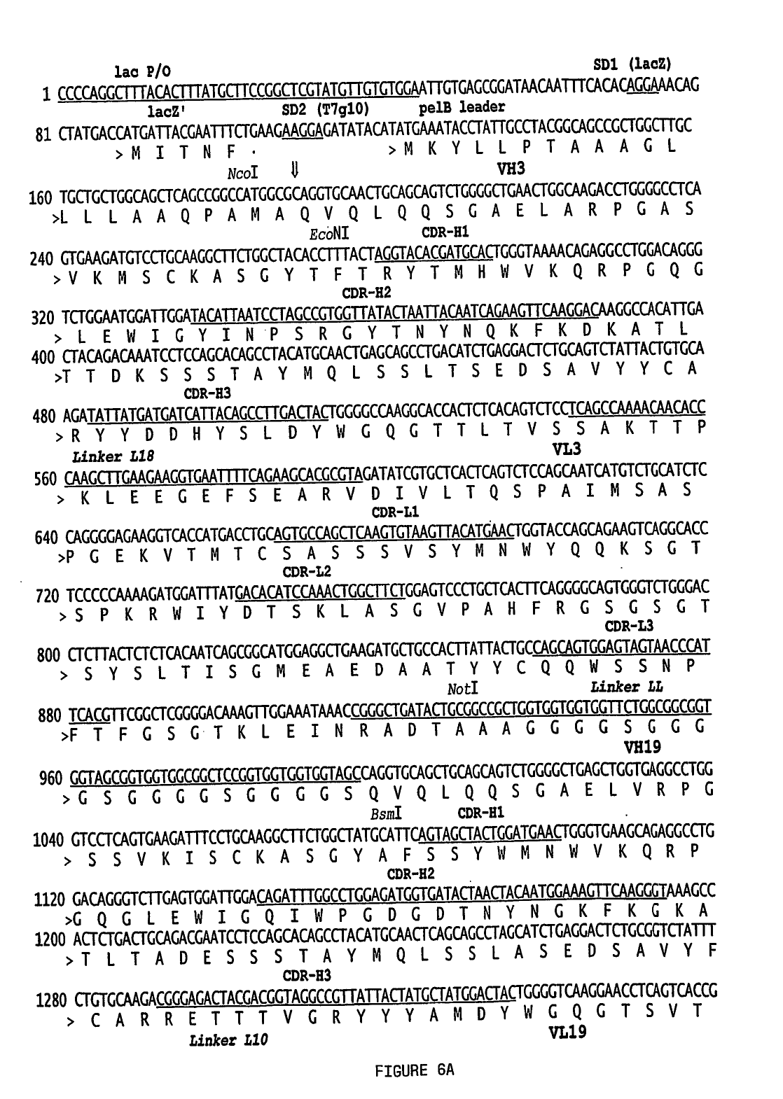

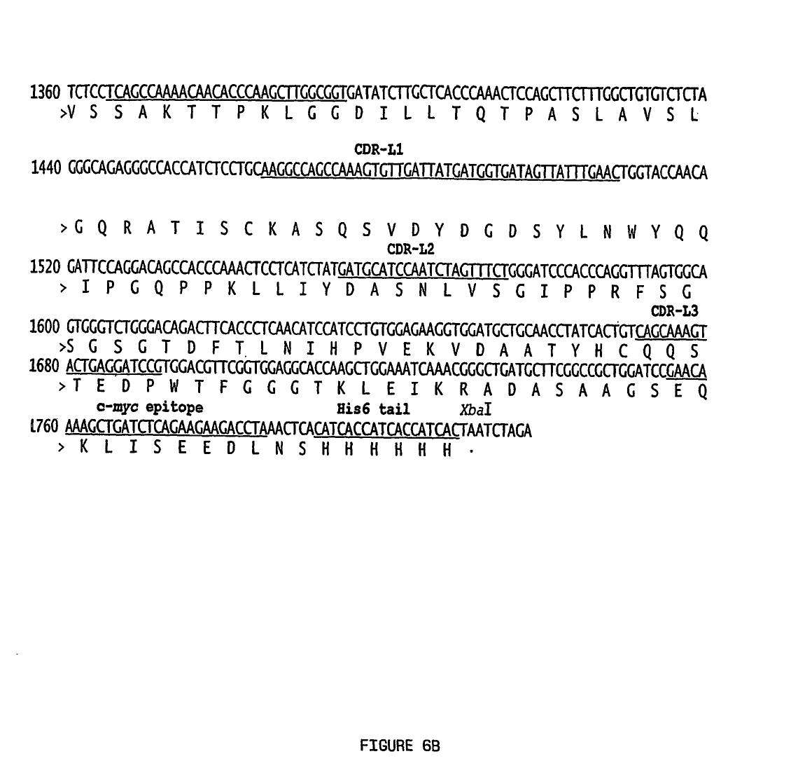

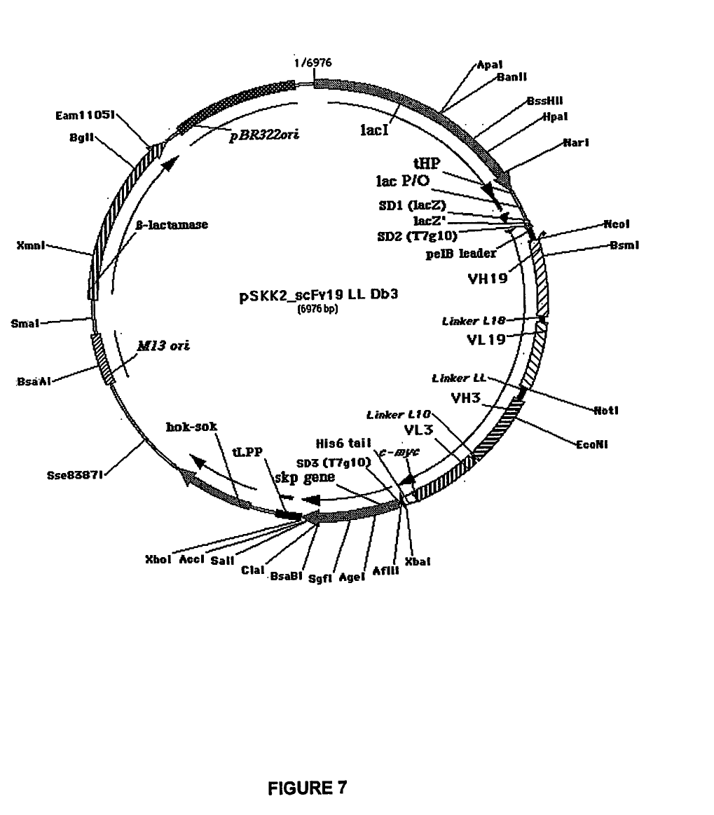

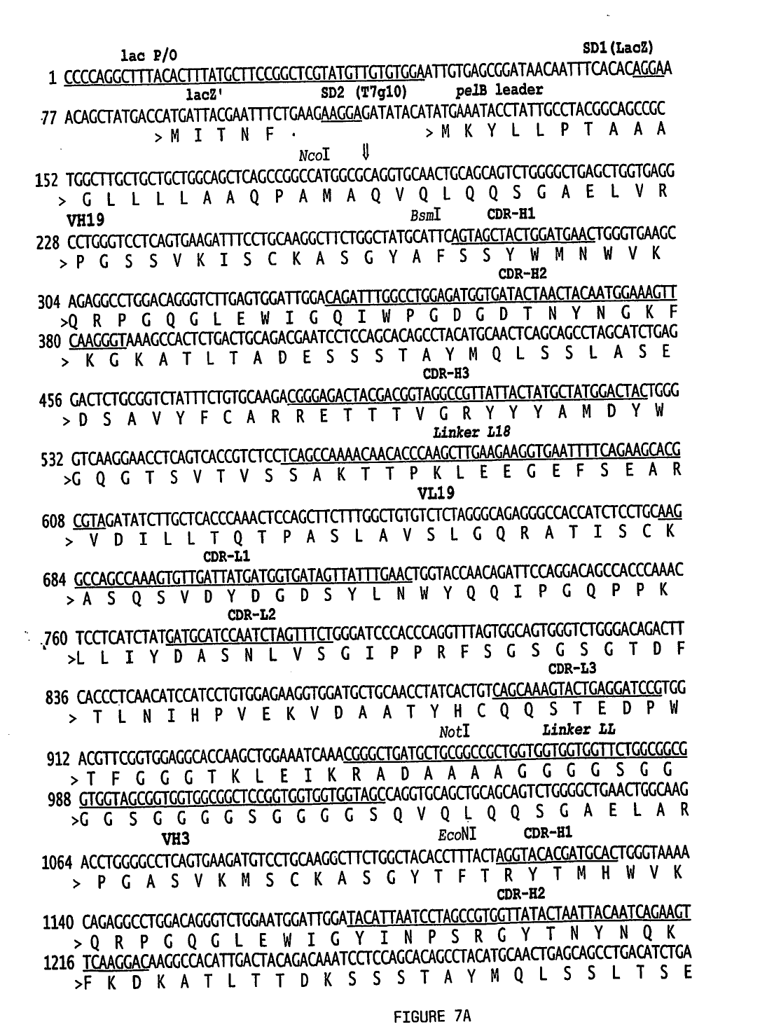

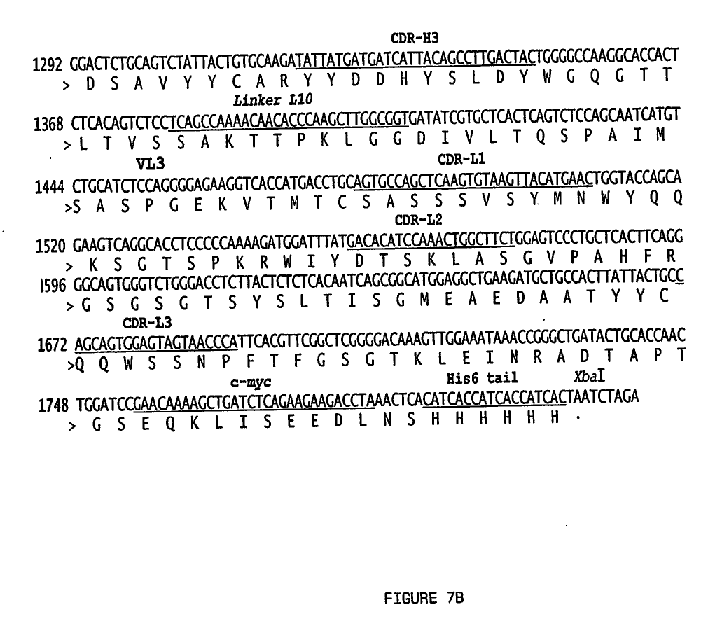

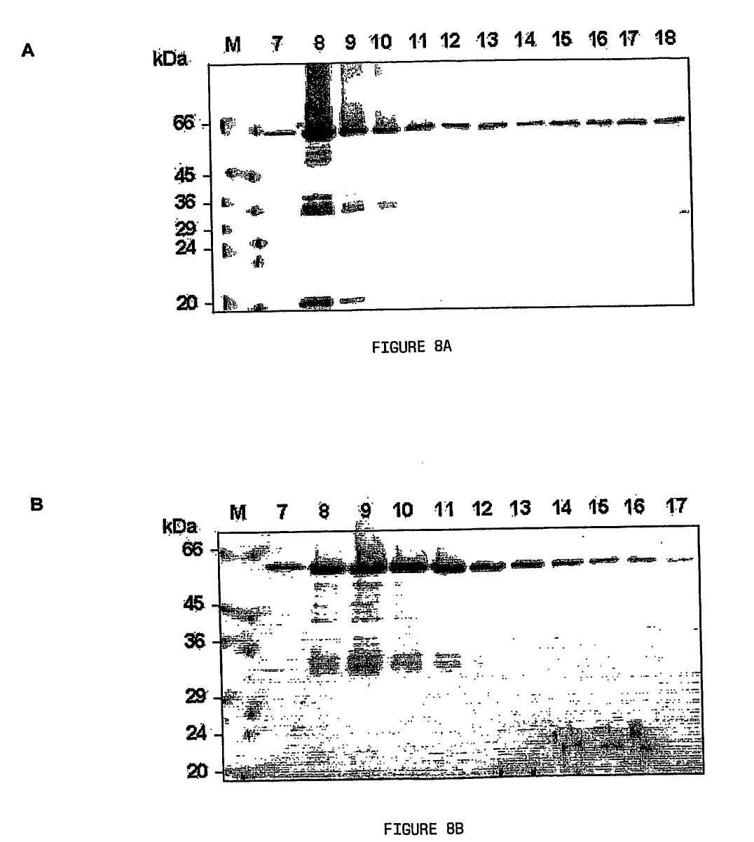

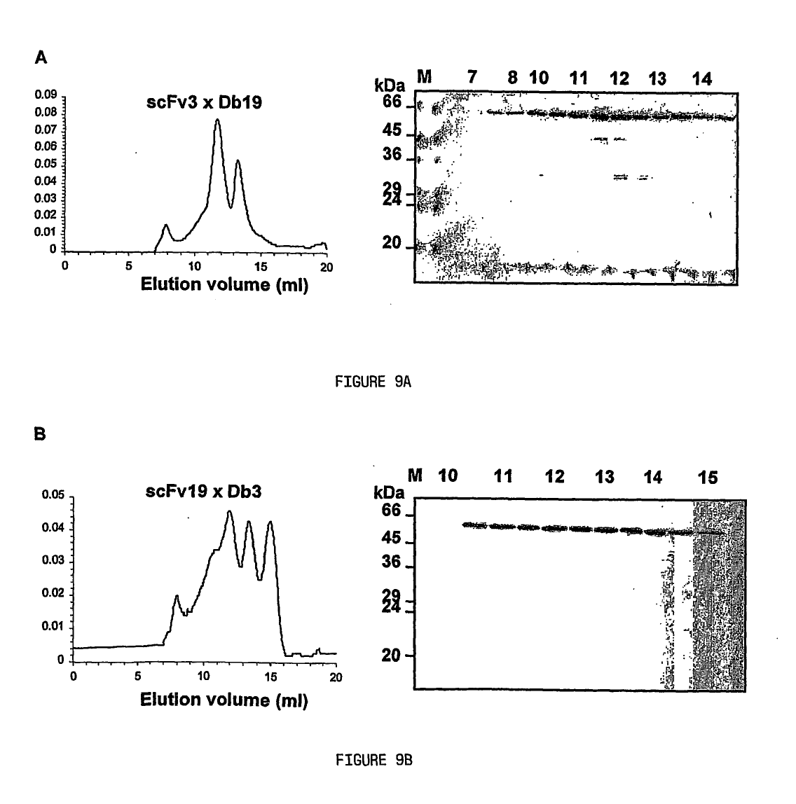

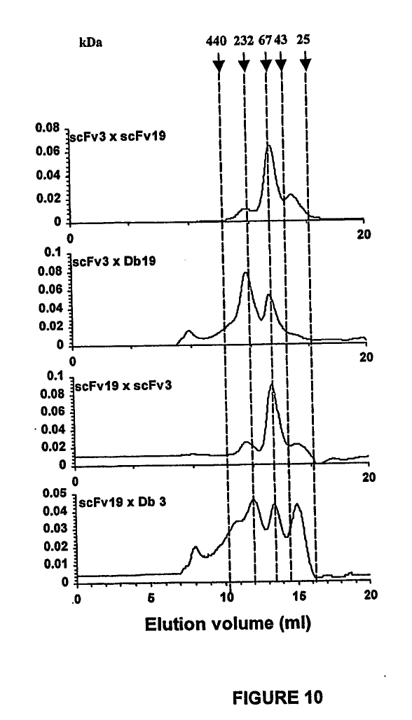

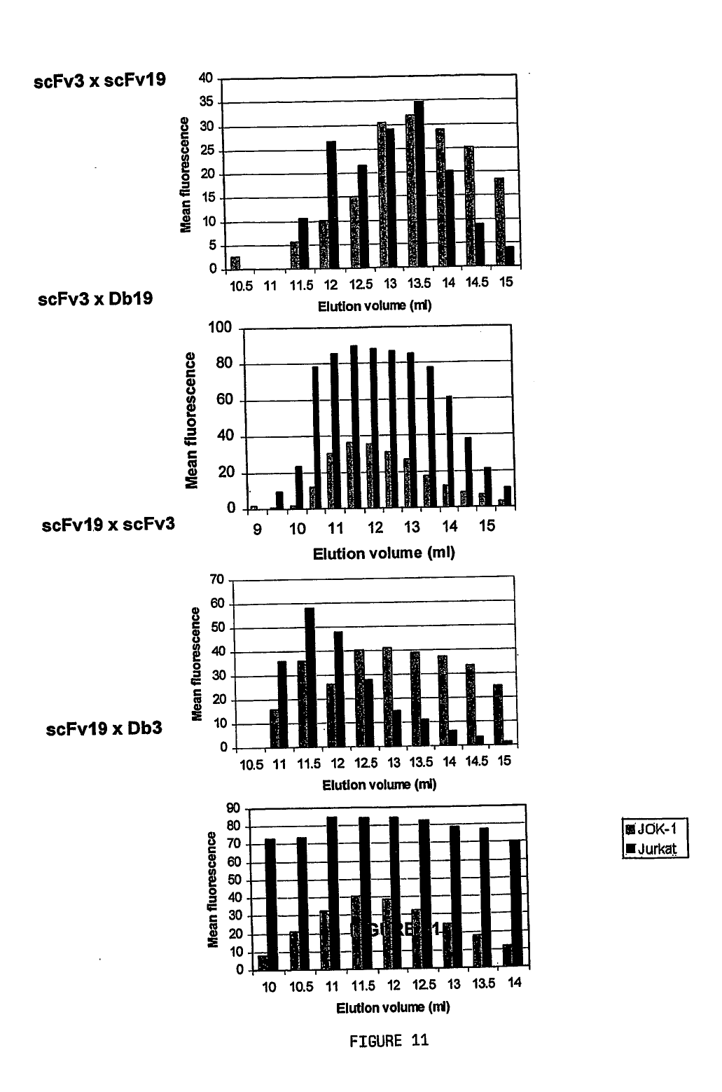

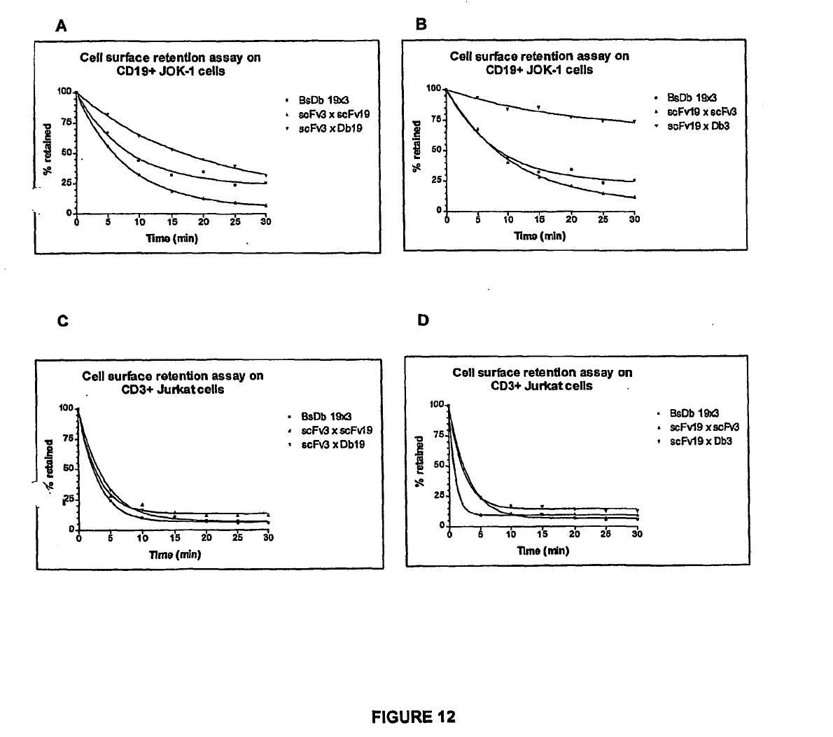

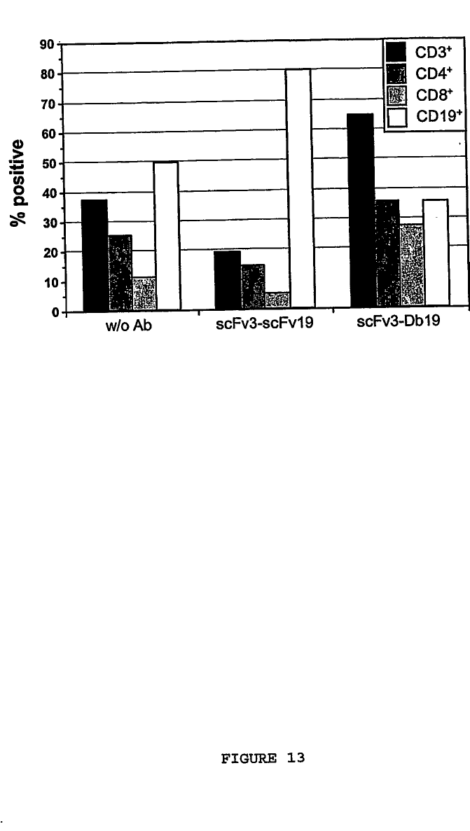

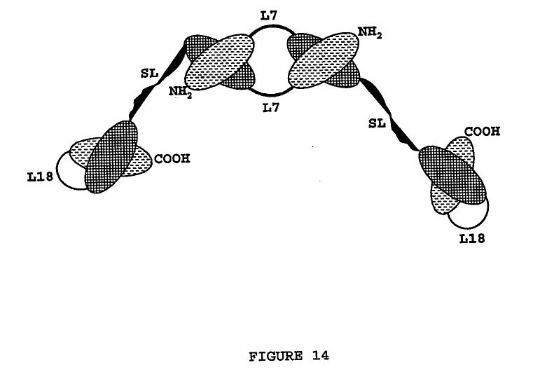

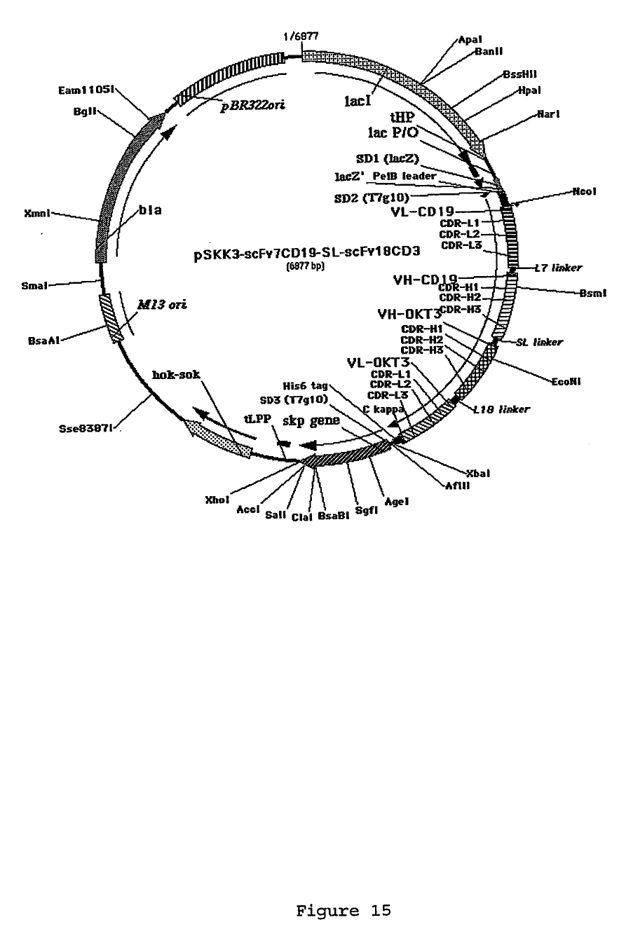

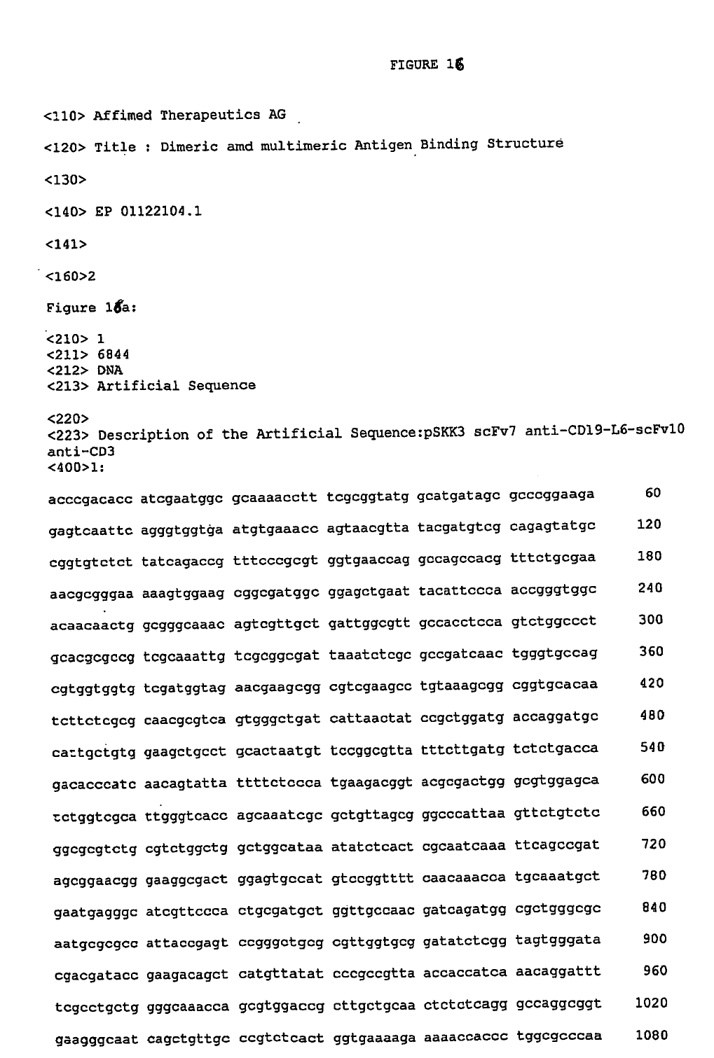

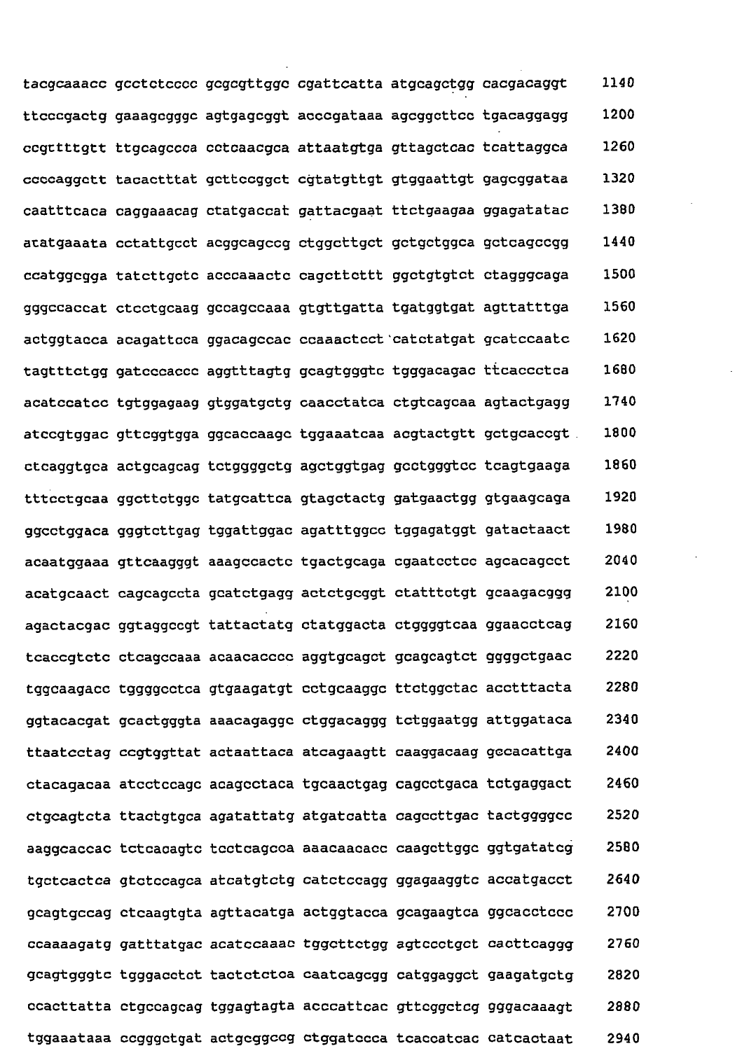

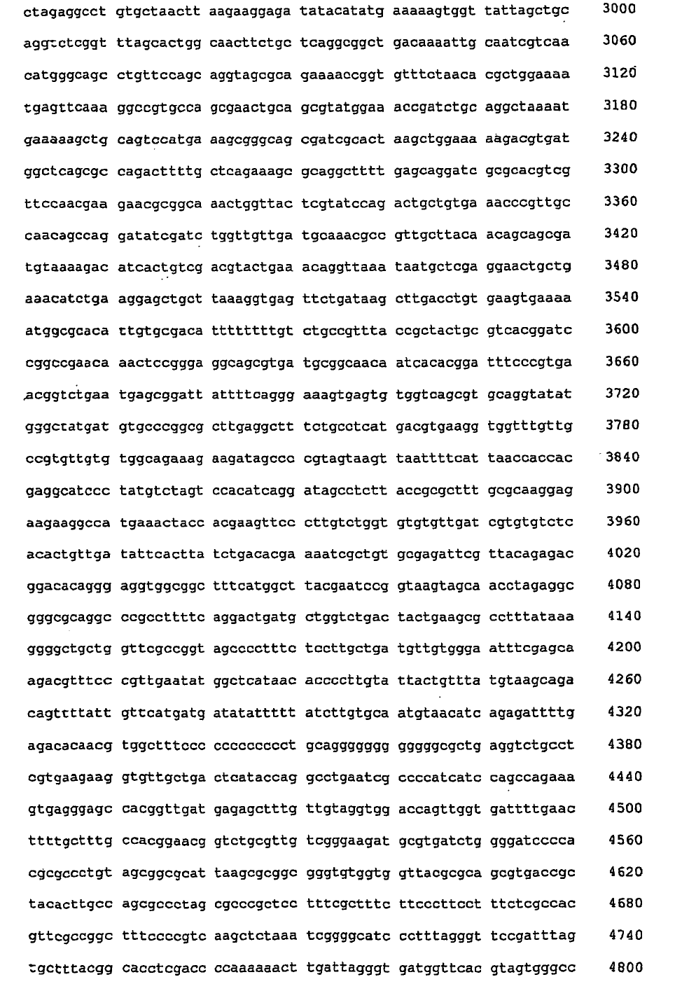

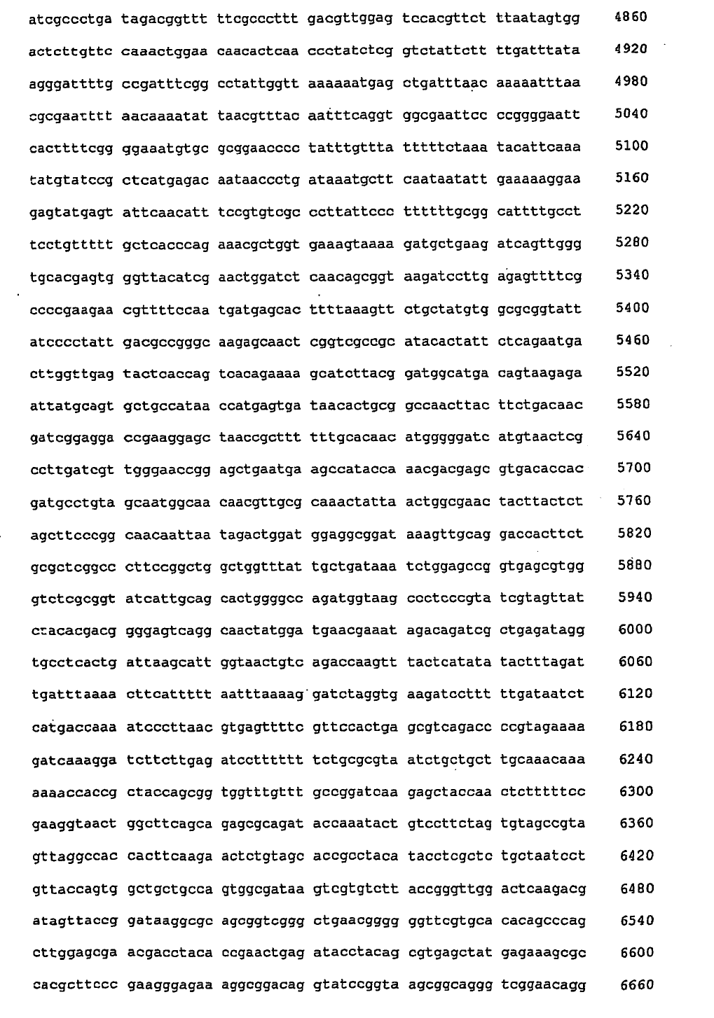

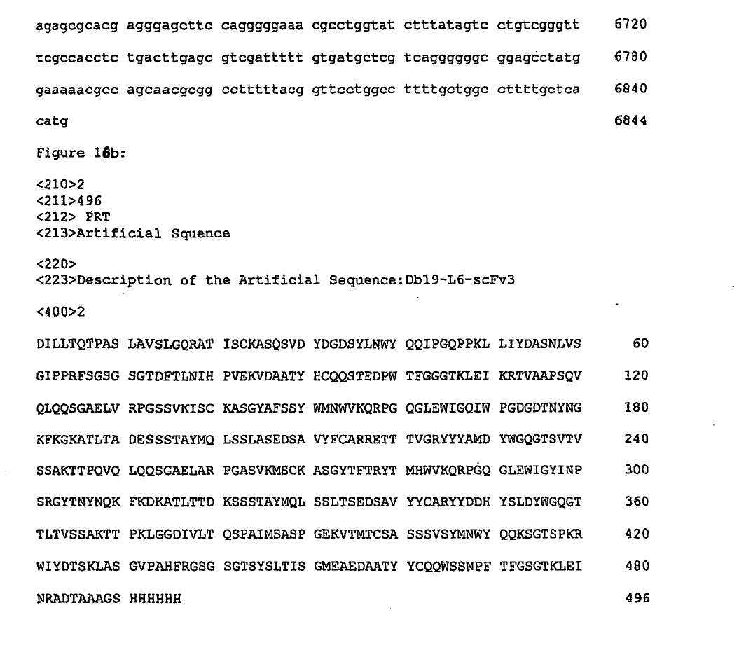

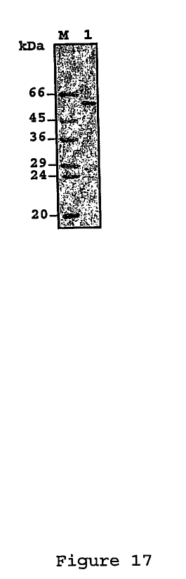

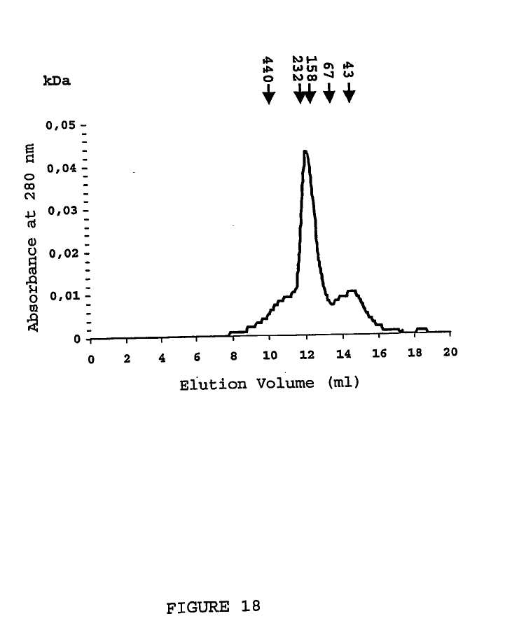

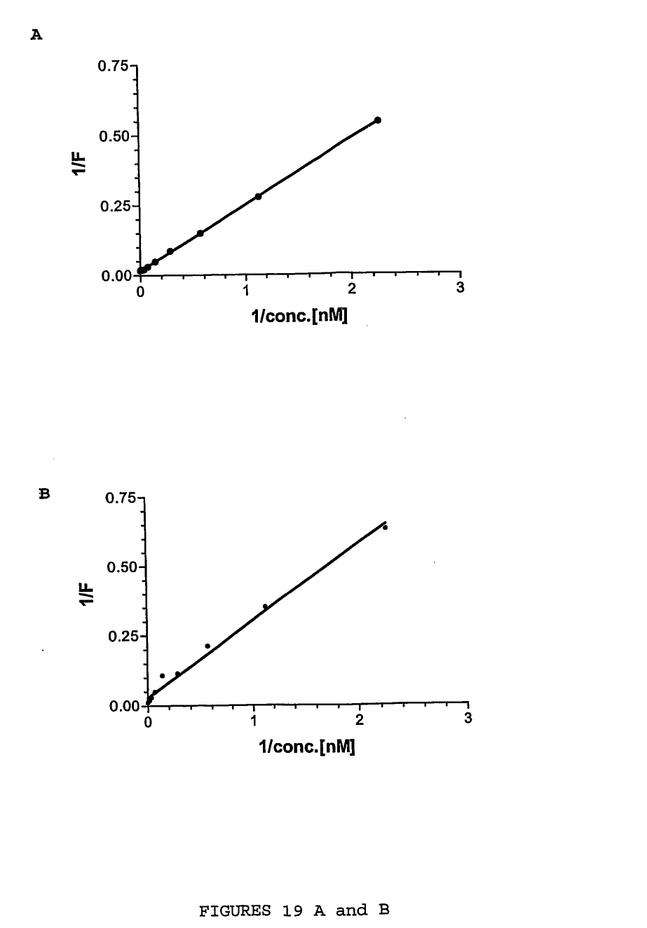

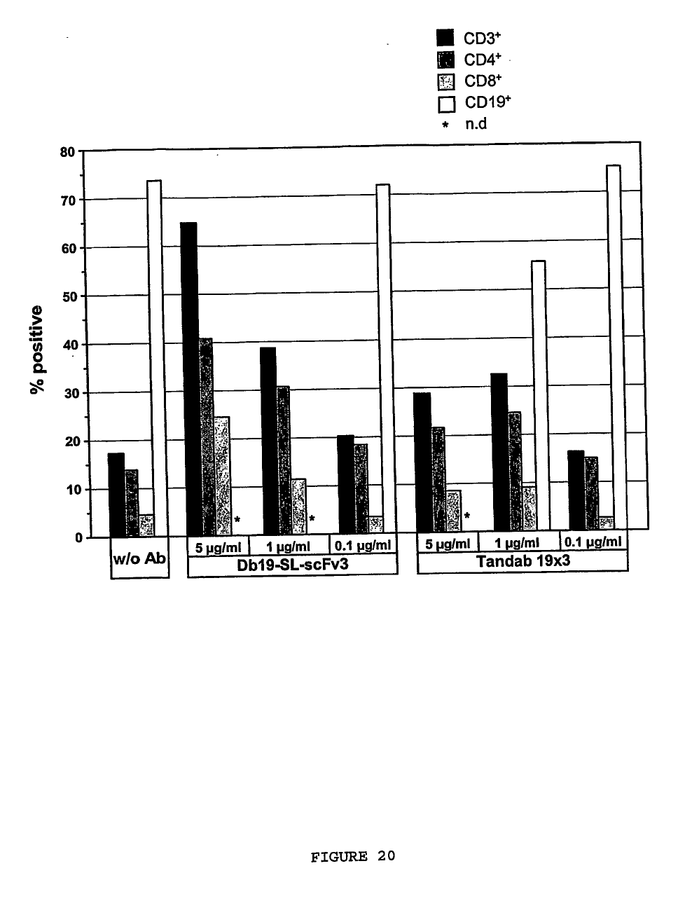

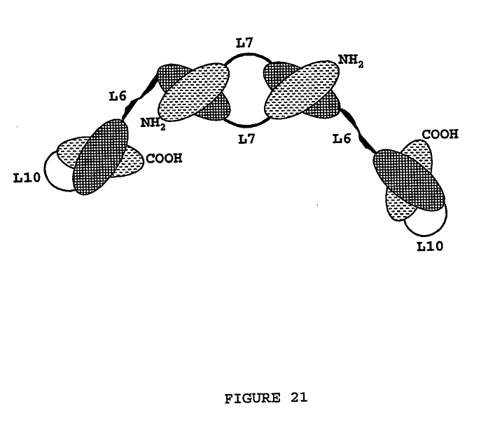

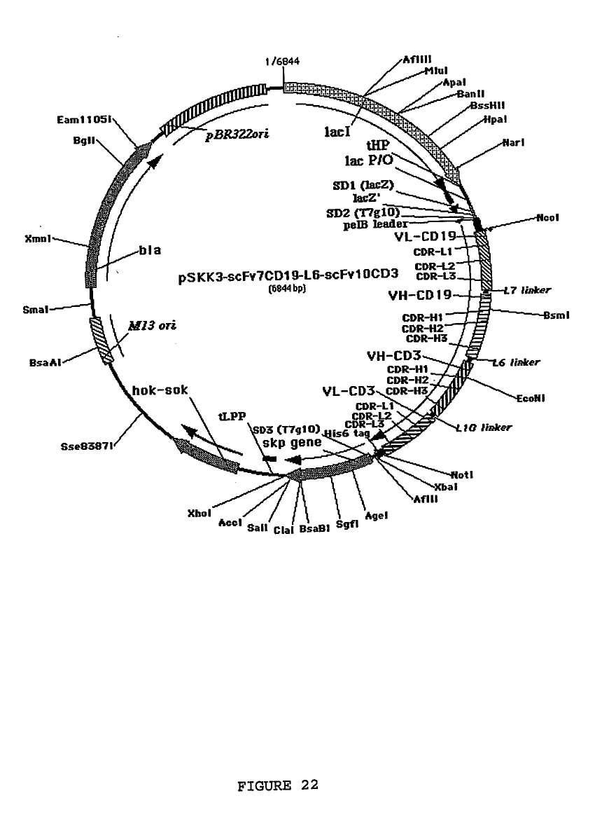

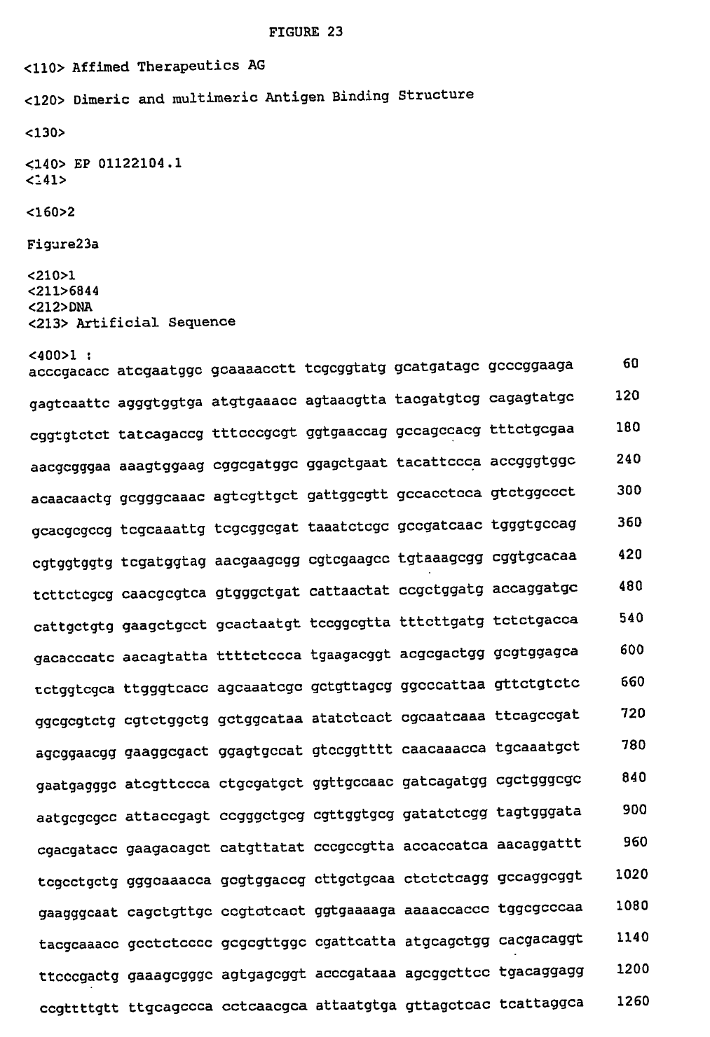

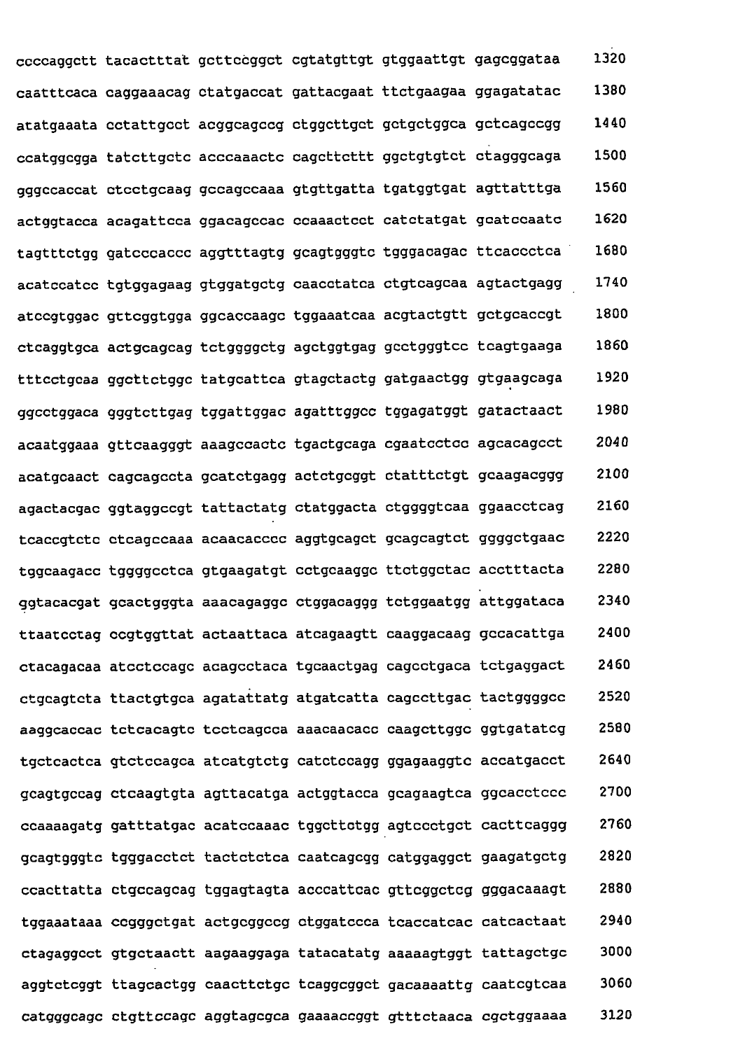

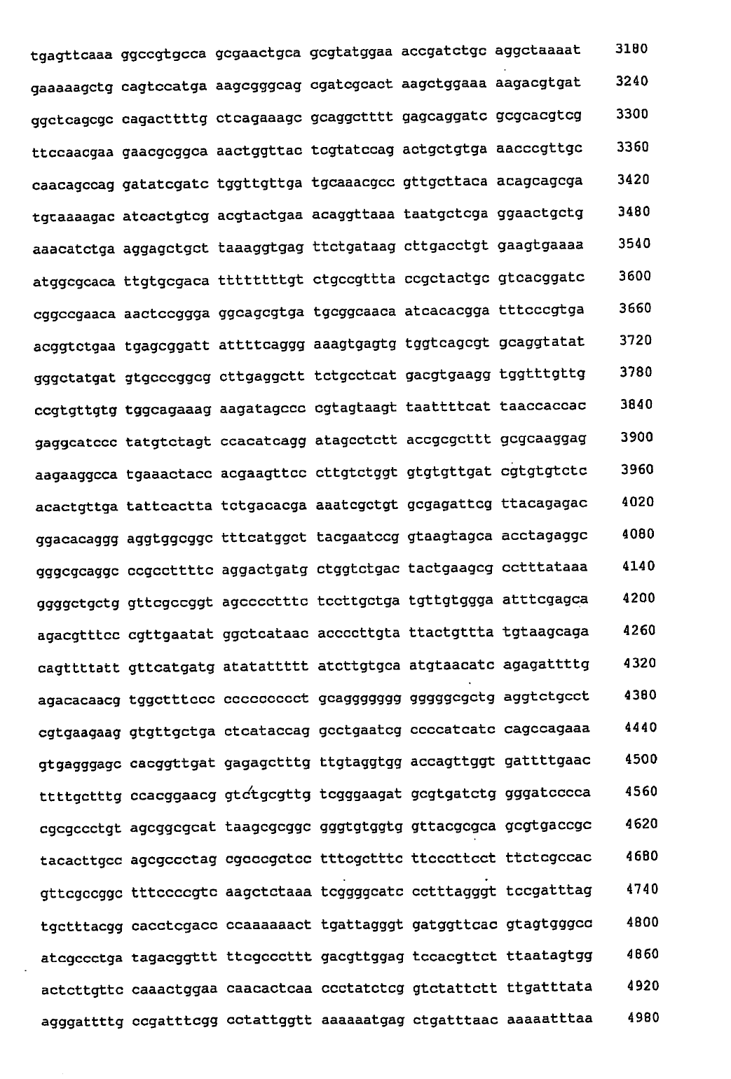

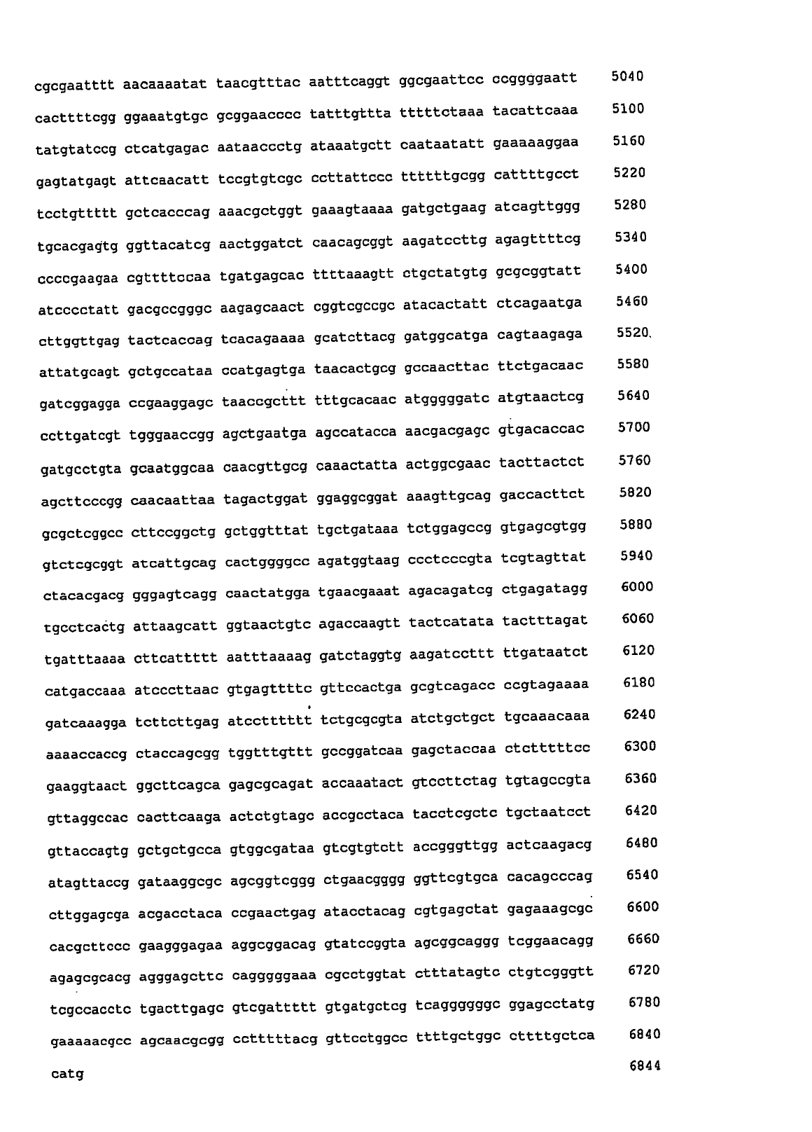

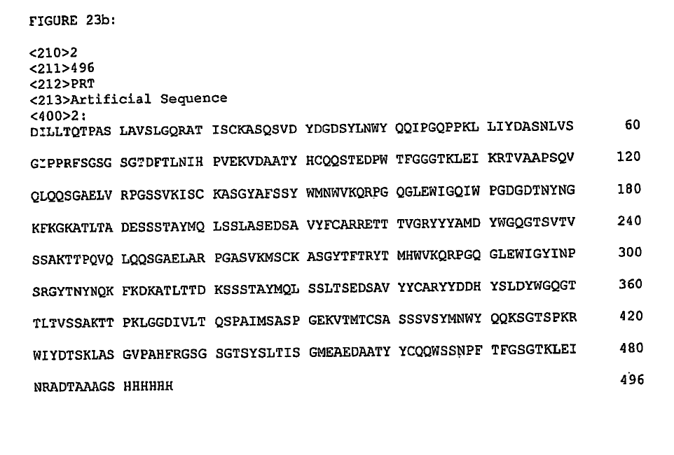

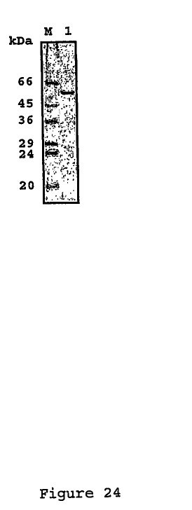

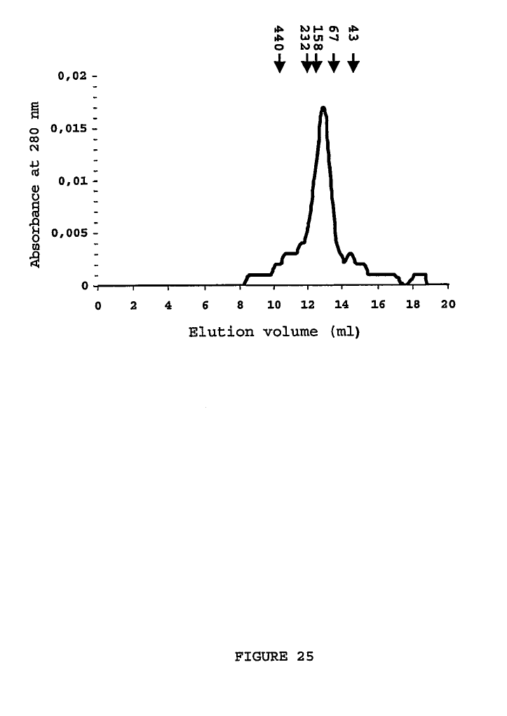

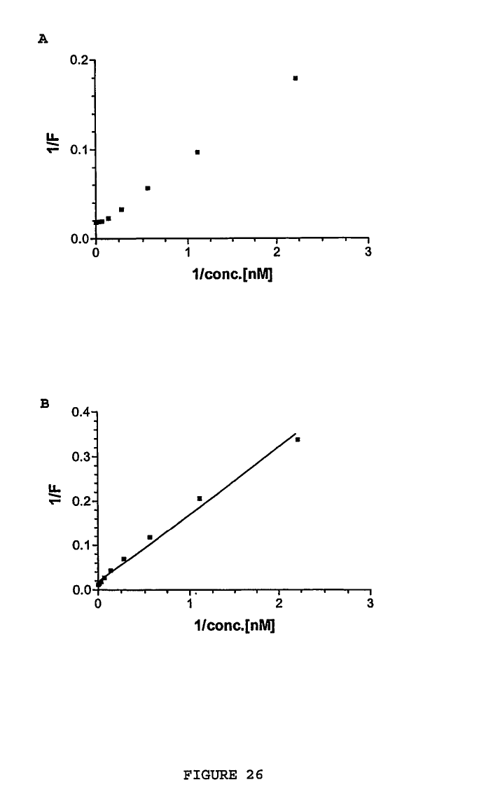

The linearized hybrid plasmid NcoI/NotI was also used for the ligation of the gene coding for the scFv_{L18}αCD19 from the plasmid pHOG HD37, and the plasmid obtained is the pHOG scFv_{L18α}CD19-LL-scFv_{L10}αCD19 (scFv19×Db19) (FIG. 4). This plasmid was linearized by PvuII/XbaI and the scFV_{L10} gene followed by a segment coding for a c-myc epitope and a hexahistidinyl tail was cut with PvuII/XbaI from the plasmid pHOG mDia OKT3. The plasmid obtained is the PHOG scFv_{L18}αCD19-LL-scFv_{L10}αCD3 (scFv19−Db3) (FIG. 5).

To increase the yield of functional antibody fragments in the bacterial periplasm, an optimized expression vector pSKK2 was generated. This vector was constructed on the base of plasmid pHKK (Horn, 1996, Appl. Microbiol. Biotechnol., 46, 524-532) containing hok/sok plasmid-free cell suicide system (Thisted et al., 1994, EMBO J., 13, 1950-1956). First, the gene coding for hybrid scFv V_{H}3-V_{L}19 was amplified by PCR from the plasmid pHOG3-19 (Kipriyanov et al., 1998, Int. J. Cancer 77, 763-772) using the primers 5-NDE, 5′-GATATACATATGAAATACCTATTGCCTACGGC (SEQ ID NO:14), and 3-AFL, 5′-CGAATTCTTAAGTTAGCACAGGCCTCTAGAGACACACAGATCTTTAG (SEQ ID NO:15). The resulting 921 bp PCR fragment was digested with NdeI and AflII, and cloned into the NdeI/AflII linearized plasmid PHKK, generating the vector pHKK3-19. To delete an extra XbaI site, a fragment of pHKK plasmid containing 3′-terminal part of the lacI gene (encodes the lac repressor), the strong transcriptional terminator t_{HP} and wild-type lac promoter/operator was amplified by PCR using primers 5-NAR, 5′-CACCCTGGCGCCCAATACGCAAACCGCC (SEQ ID NO:16), and 3-NDE, 5′-GGTATTTCATATGTATATCTCCTTCTTCAGAAATTCGTAATCATGG (SEQ ID NO:17). The resulting 329 bp DNA fragment was digested with NarI and NdeI, and cloned into NarI/NdeI linearized plasmid pHKK3-19 generating the vector pHKKΔXba. To introduce a gene encoding the Skp/OmpH periplasmic factor for higher recombinant antibody production (Bothmann and Plückthun, 1998, Nat. Biotechnol., 16, 376-380), the skp gene was amplified by PCR with primers skp-3, 5′-CGAATTCTTAAGAAGGAGATATACATATGAAAAAGTGGTTATTAGCTGCAGG (SEQ ID NO:18) and skp-4, 5′-CGAATTCTCGAGCATTATTTAACCTGTTTCAGTACGTCGG (SEQ ID NO:19) using as a template the plasmid pGAH317 (Holck and Kleppe, 1988, Gene, 67, 117-124). The resulting 528 bp PCR fragment was digested with AflII and XhoI and cloned into the AflII/XhoI digested plasmid pHKKΔXba resulting in the expression plasmid pSKK2.

The plasmids pHOG scFv_{L18}αCD3-LL-scFv_{L10}αCD19 (scFv3−Db19) and pHOG scFv_{L18}αCD19-LL-scFv_{L10}αCD3 (scFv19−Db3) were cut by NcoI/XbaI, and ligated in the NcoI/XbaI linearized plasmid pSKK2. The resulting plasmids are pSKK2 scFv_{L18}αCD3-LL-scFv_{L10}αCD19 and pSKK2 scFv_{L18}αCD19-LL-scFv_{L10}αCD3. The complete nucleotide and amino acid sequences are given in FIGS. 6 and 7, respectively.

### Example 2

### Expression and Purification of the Multimeric Fv Molecules in Bacteria

The E. coli K12 strain RV308 (Maurer et al., 1980, J. Mol. Biol. 139, 147-161), transformed with the expression plasmids pSKK2 scFv_{L18}αCD3-LL-scFv_{L10}αCD19 and pSKK2 scFv_{L18}αCD19-LL-scFv_{L10}αCD3, was grown overnight in 2xYT medium with 50 μg/ml ampicillin and 100 mM glucose (2xYT_{GA}) at 28° C. Dilutions (1:50) of the overnight cultures in 2xYT_{GA} were grown as flask cultures at 28° C. with shaking at 200 rpm. When cultures reached OD₆₀₀=0.8, bacteria were pelleted by centrifugation at 5,000×g for 10 min at 20° C., and resuspended in the same volume of fresh YTBS medium (2xYT containing 1 M sorbitol and 2.5 mM glycine betaine; Blacwell & Horgan, 1991, FEBS Letters. 295, 10-12) containing 50 μg/ml ampicillin. IPTG was added to a final concentration of 0.2 mM, and growth was continued at 20° C. for 18-20 h. Cells were harvested by centrifugation at 9,000×g for 20 min and 4° C. To isolate soluble periplasmic proteins, the pelleted bacteria were resuspended in 5% of the initial volume of ice-cold 50 mM Tris-HCl, 20% sucrose, and 1 mM EDTA, pH 8.0. After a 1 h incubation on ice with occasional stirring, the spheroplasts were centrifuged at 30,000×g for 30 min at 4° C., leaving the soluble periplasmic extract as the supernatant and spheroplasts plus the insoluble periplasmic material as the pellet. The periplasmic fractions were dialyzed against start buffer (50 mM Tris-HCl, 1 M NaCl, 50 mM Imidazole, pH 7.0) at 4° C. The dialyzed solution containing recombinant product was centrifuged at 30,000×g for 30 min at 4° C. The recombinant product was concentrated by ammonium sulfate precipitation (final concentration 70% of saturation). The protein precipitate was collected by centrifugation (10,000×g, 4° C., 40 min), and dissolved in 10% of the initial volume of 50 mM Tris-HCl, 1 M NaCl, pH 7.0. Immobilized metal affinity chromatography (IMAC) was performed at 4° C. using a 5 ml column of Chelating Sepharose (Pharmacia) charged with Cu²⁺ and equilibrated with 50 mM Tris-HCl, 1 M NaCl, pH 7.0 (start buffer). The sample was loaded by passing the sample over the column. It was then washed with twenty column volumes of start buffer followed by start buffer containing 50 mM imidazole until the absorbency (280 nm) of the effluent was minimal (about thirty column volumes). Absorbed material was eluted with 50 mM Tris-HCl, 1 M NaCl, 250 mM imidazole, pH 7.0. The elution fractions containing the multimeric Fv-molecules were identified by Western-blot analysis using anti-c-myc Mab 9E10, performed as previously described (Kipriyanov et al., 1994, Mol. Immunol. 31, 1047-1058) and as illustrated in FIG. 8A for scFv3−Db19 and FIG. 8B for scFv19−Db3.

The positive fractions were collected and concentrated on an Ultrafree-15 centrifugal filter device (Millipore Corporation, Eschborn, Germany) until 0.5 ml was collected.

Further purification of the multimeric Fv-molecules was done by size-exclusion FPLC on a Superdex 200 HR10/30 column (Pharmacia) in PBSI (15 mM sodium phosphate, 0,15 M NaCl, 50 mM Imidazole, pH 7.0). Sample volumes for preparative chromatography were 500 μl, and the flow rate was 0.5 ml/min, respectively. The column was calibrated with High and Low Molecular Weight Gel Filtration Calibration Kits (Pharmacia). The elution fractions containing the multimeric Fv-molecules were identified by Western-blot analysis using anti-c-myc Mab 9E10 performed as previously described (Kipriyanov et al., 1994, Mol. Immunol. 31, 1047-1058), and the results are presented in FIGS. 9A and 9B for scFv3−Db19 and scFv19−Db3 molecules, respectively. The fractions were collected and stored individually on ice.

The generated Fv molecules were compared with two scFv-scFv tandems, scFv3−scFv19 and scFv19−scFv3 (FIG. 10), produced and purified under the same conditions. FIG. 10 clearly demonstrates that higher molecular forms were obtained for the scFv3×Db 19 and scFv 19×Db3 in comparison with scFv3×scFv 19 and scFv 19×scFv3. The main peak for scFv3−scFv19 and scFv19−scFv3 molecules correspond to a molecular weight of about 67 kDa, and to about 232 kDa for the scFv3−Db19 and scFv19−Db3. The presence of the dimerization motif on the C-terminus of the molecule has a positive effect for the multimerisation of the molecules.

### Example 3

### Characterization of the Multimeric Fv Molecules by Flow Cytometry

The human CD3⁺/CD19⁻ acute T cell leukemia line Jurkat and the CD19⁺/CD3⁻B cell line JOK-1 were used for flow cytometry. In brief, 5×10⁵ cells in 50 μl RPMI 1640 medium (GIBCO BRL, Eggenstein, Germany) supplemented with 10% FCS and 0.1% sodium azide (referred to as complete medium) were incubated with 100 μl of a multimeric Fv molecule preparation for 45 min on ice. After washing with complete medium, the cells were incubated with 100 μl of 10 μg/ml anti c-myc MAb 9E10 (IC Chemikalien, Ismaning, Germany) in the same buffer for 45 min on ice. After a second washing cycle, the cells were incubated with 100 μl of FITC-labeled goat anti-mouse IgG (GIBCO BRL) under the same conditions as before. The cells were then washed again and resuspended in 100 μl of 1 μg/ml solution of propidium iodide (Sigma, Deisenhofen, Germany) in complete medium to exclude dead cells. The relative fluorescence of stained cells was measured using a FACScan flow cytometer (Becton Dickinson, Mountain View, Calif.) or Epics XL flow cytometer systems (Beckman Coulter, Miami, Fla.).

Flow cytometry experiments demonstrated specific interactions with both human CD3⁺Jurkat and the CD19⁺JOK-1 cells for all the multimeric Fv-molecules (FIG. 11).

For the CD19 and CD3 binding affinities, we decided to use the fractions corresponding to the monomers for scFv3×scFv 19 and scFv19−scFv3, and to the multimers for scFv3×Db 19 and scFv 19×Db3.

### Example 4

### In Vitro Cell Surface Retention Assay of the Multimeric Fv Molecules

Cell surface retention assays were performed at 37° C. essentially as described (Adams et al., 1998, Cancer Res. 58, 485-490), except that the detection of the retained antibody fragments was performed using anti c-myc MAb 9E10 followed by FITC-labeled anti-mouse IgG. Kinetic dissociation constant (k_{off}) and the half-life (t_{1/2}) for dissociation of the multimeric Fv-molecules were deduced from a one-phase exponential decay fit of experimental data using “GraphPad” Prism (GraphPad Software, San Diego, Calif.). For control, the bispecific diabody CD19×CD3 (BsDb 19×3) described previously (Kipriyanov et al., 1998, Int. J. Cancer 77, 763-777; Cochlovius et al., 2000, J. Immunol. 165, 888-895) was used. The results of the experiments are shown in FIG. 12 and summarized in Table 1.

The scFv3−scFv19 had a relatively short retention half-life (t_{1/2}) on CD19⁺JOK-1 cells, almost two time less than with the t_{1/2} of the BsDb 19×3 (Table 1). In contrast, the scFv3−Db19 was retained longer on the surface of JOK-1 cells. For the scFv19−scFv3, the t_{1/2} is in the same range as the t_{1/2} of the BsDb 19×3. The retention of the scFv3−Db19 is significantly higher, with t_{1/2}=65.71 min, in comparison with the others molecules (Table 1). The half-lives of all the multispecific Fv-molecules on the surface of CD3⁺Jurkat cells were relatively short. The length of the linker appeared to have some influence on antigen binding, since the scFv3−Db19 and scFv19−Db3 showed a significantly slower k_{off} for CD19-positive cells than for the BsDb 19×3, scFv3×scFv 19 and scFv19−scFv3.

TABLE 1
Binding kinetics of recombinant bispecific molecules

| | k_{off} | t_{1/2} | | k_{off} (s⁻¹/ | t_{1/2} |
|---|---|---|---|---|---|
| Antibody | (s⁻¹/10⁻³) | (min) | Antibody | 10⁻³) | (min) |
| A. JOK-1 cells (CD3⁻/CD19⁺) | | | B. JOK-1 cells (CD3⁻/CD19⁺) | | |
| BsDb 19x3 | 0.9945 | 11.62 | BsDb 19x3 | 0.1512 | 10.68 |
| scFv3-scFv19 | 1.814 | 6.368 | scFv19-scFv3 | 0.05547 | 8.622 |
| scFv3-Db19 | 0.6563 | 17.6 | scFv19-Db3 | 0.01171 | 65.71 |
| C. Jurkat cells (CD3⁺/CD19⁻) | | | D. Jurkat cells (CD3⁺/CD19⁻) | | |
| BsDb 19x3 | 4.268 | 2.707 | BsDb 19x3 | 4.268 | 2.707 |
| scFv3-scFv19 | 2.912 | 3.967 | scFv19-scFv3 | 6.91 | 1.672 |
| scFv3-Db19 | 3.161 | 3.655 | scFv19-Db3 | 3.4 | 3.394 |

### Example 5

### In Vitro Analysis of Anti-Tumor Activity of Recombinant Multivalent Molecules

Freshly isolated peripheral blood mononuclear cells (PBMC) from a patient with chronic lymphocytic leukemia (CLL) were seeded in individual wells of a 12-well plate in 2 ml RPMI-Medium/10% FCS (Invitrogen, Breda, The Netherlands) at a density of 2×10⁶ cells/ml. The recombinant antibodies scFv3−scFv19 and scFv3−Db19 were added at concentration of 5 μg/ml. After a 5 day incubation, the cells were harvested, counted, and stained with anti-CD3 MAb OKT3 (DKFZ, Heidelberg, Germany), anti-CD4 MAb Edu-2 (Chemicon, Hofheim, Germany), anti-CD8 MAb UCH-T4 (Chemicon, Hofheim, Germany), and anti-CD19 MAb HD37 (DKFZ, Heidelberg, Germany) for flow cytometric analysis. 10⁴ living cells were analyzed using a Beckman-Coulter flow cytometer and the relative and absolute amounts of CD3⁺, CD4⁺, CD8⁺ and CD19⁺ cells were plotted.

The results shown in FIG. 13 demonstrated that tetravalent scFv3−Db19 molecules caused vigorous proliferation of autologous T cells and killing of C19⁺ tumor cells. In contrast, bivalent scFv3−scFv19 molecules had nearly no effect.

### Example 6

### Construction of the Plasmid pSKK3-scFv_{L7}Anti-CD19-SL-scFv_{L18}Anti-CD3 for the Expression of Multimeric Fv-Antibody (Db19-SL-scFv3) in Bacteria

For generation of multimeric Fv constructs, the plasmids pHOG_HD37, pHOG_Dia_HD37, pHOG_mOKT3+NotI, and pHOG_Dia_mOKT3 encoding the antibody fragments derived either from hybridoma HD37 specific to human CD19 (Kipriyanov et al., 1996, J. Immunol. Meth. 196, 51-62; Le Gall et al., 1999, FEBS Lett., 453, 164-168) or from hybridoma OKT3 specific to human CD3 (Kipriyanov et al., 1997, Protein Eng. 10, 445-453) were used.

To generate a gene encoding the anti-CD19 scFv_{L7} with the V_{L}-V_{H} orientation, the V_{L}-HD37 gene was amplified by PCR using as a template the plasmid DNA pHOG_HD37 (Kipriyanov et al., 1996, J. Immunol. Meth. 196, 51-62) and primers VL_Nco, 5′-CAGCCGGCCATGGCGGATATCTTGCTCACCCAAACTCCAGC (SEQ ID NO:20) and 3_Ck, 5′-AGACGGTGCAGCAACAGTACGTTTGATTTCCAGC (SEQ ID NO:21). The resulting 371 bp PCR fragments code for the anti-CD19 VL domain followed by 7 amino acid Arg-Thr-Val-Ala-Ala-Pro-Ser linker. In turn, the V_{H}-HD37 gene was amplified by PCR using as a template the plasmid DNA pHOG_HD37 (Kipriyanov et al., 1996, J. Immunol. Meth. 196, 51-62) and primers 5_Ck, 5′-CGTACTGTTGCTGCACCGTCTCAGGTGCAACTGCAGCAGTC (SEQ ID NO:22) and VH_Not, 5′-GAAGATGGATCCAGCGGCCGCTGAGGAGACGGTGACTGAGGTTCC (SEQ ID NO:23). The resulting 416 bp PCR fragment codes for the anti-CD19 VH domain preceded by 7 amino acid Arg-Thr-Val-Ala-Ala-Pro-Ser linker. The whole gene for anti-CD19 scFv_{L7} was assembled by PCR from 371 bp and 416 bp DNA fragments using primers VL_Nco and VH_Not. The resulting 764 bp PCR fragment was digested with NcoI and NotI and cloned into NcoI/NotI-linearized plasmid pDISC2/SL (Kipriyanov et al., 1999, J. Mol. Biol. 293, 41-56), thus generating the plasmid pDISC-scFv_{L7}anti-CD19-SL-scFV_{L10}anti-CD3.

To increase the yield of functional scFv-antibodies in the bacterial periplasm, an optimized expression vector pSKK3 was generated (FIG. 15). This vector was constructed on the basis of plasmid pHKK (Horn et al., 1996, Appl. Microbiol. Biotechnol. 46, 524-532) containing hok/sok plasmid-free cell suicide system (Thisted et al., 1994, EMBO J. 13, 1960-1968). First, the gene coding for hybrid scFv V_{H}3-V_{L}19 was amplified by PCR from the plasmid pHOG3-19 (Kipriyanov et al., 1998, Int. J. Cancer 77, 763-772) using the primers 5-NDE, 5′-GATATACATATGAAATACCTATTGCCTACGGC, (SEQ ID NO:24) and 3-AFL, 5′-CGAATTCTTAAGTTAGCACAGGCCTCTAGAGACACACAGATCTTTAG (SEQ ID NO:25). The resulting 921 bp PCR fragment was digested with NdeI and AflII and cloned into the NdeI/AflII linearized plasmid pHKK, generating the vector pHKK3-19. To delete an extra XbaI site, a fragment of the PHKK plasmid containing the 3′-terminal part of the lacI gene (encoding the lac repressor), the strong transcriptional terminator tHP, and wild-type lac promoter/operator was amplified by PCR using primers 5-NAR, 5′-CACCCTGGCGCCCAATACGCAAACCGCC, (SEQ ID NO:16) and 3-NDE, 5′-GGTATTTCATATGTATATCTCCTTCTTCAGAAATTCGTAATCATGG (SEQ ID NO:17). The resulting 329 bp DNA fragment was digested with NarI and NdeI and cloned into NarI/NdeI-linearized plasmid pHKK3-19, generating the vector pHKKΔXba. To introduce a gene encoding the Skp/OmpH periplasmic factor for higher recombinant antibody production (Bothmann and Plückthun, 1998, Nat. Biotechnol. 16, 376-380), the skp gene was amplified by PCR with primers skp-3, 5′-CGAATTCTTAAGAAGGAGATATACATATGAAAAAGTGGTTATTAGCTGCAGG (SEQ ID NO:18), and skp-4, 5′-CGAATTCTCGAGCATTATTTAACCTGTTTCAGTACGTCGG (SEQ ID NO:19), using as a template the plasmid pGAH317 (Holck and Kleppe, 1988, Gene 67, 117-124). The resulting 528 bp PCR fragment was digested with AflII and XhoI and cloned into the AflII/XhoI digested plasmid pHKKΔXba resulting in the expression plasmid pSKK2.

For removing the sequence encoding the potentially immunogenic c-myc epitope, the NcoI/XbaI-linearized plasmid pSKK2 was used for cloning the NcoI/XbaI-digested 902 bp PCR fragment encoding the scFv phOx31E (Marks et al., 1997, BioTechnology 10, 779-783), which was amplified with primers DP1 and His-Xba, 5′-CAGGCCTCTAGATTAGTGATGGTGATGGTGATGGG (SEQ ID NO:26). The resulting plasmid pSKK3 was digested with NcoI and NotI and used as a vector for cloning the gene coding for anti-CD3 scFv₁₈, which was isolated as a 751 bp DNA fragment after digestion of plasmid pHOG21_dmOKT3+NotI (Kipriyanov et al., 1997, Protein Eng. 10, 445-453) with NcoI and NotI. The resulting plasmid pSKK3_scFv_{L18}anti-CD3 was used as a template for PCR amplification of the gene encoding the anti-CD3 scFv₁₈ with primers Bi3h, 5′-CCGGCCATGGCGCAGGTGCAGCTGCAGCAGTCTGG (SEQ ID NO:27), and P-skp 5′-GCTGCCCATGTTGACGATTGC (SEQ ID NO:28). The generated 919 bp PCR fragment was digested with PvuII and XbaI and cloned into PvuII/XbaI-cut plasmid pDISC-scFv_{L7}anti-CD19-SL-scFv_{L10}anti-CD3. The resulting plasmid pDISC-scFv_{L7}anti-CD19-SL-scFv_{L18}anti-CD3 was digested with NcoI and XbaI, and the 1536 bp DNA fragment was isolated and cloned into NcoI/XbaI-linearized vector pSKK3.

The generated plasmid pSKK3-scFv_{L7}anti-CD19-SL-scFv_{L18}anti-CD3 (FIG. 15) contains several features that improve plasmid performance and lead to increased accumulation of functional bivalent product in the E. coli periplasm under conditions of both shake-flask cultivation and high cell density fermentation. These are the hok/sok post-segregation killing system, which prevents plasmid loss, strong tandem ribosome-binding sites, and a gene encoding the periplasmic factor Skp/OmpH that increases the functional yield of antibody fragments in bacteria. The expression cassette is under the transcriptional control of the wt lac promoter/operator system and includes a short sequence coding for the N-terminal peptide of β-galactosidase (lacZ′) with a first rbs derived from the E. coli lacZ gene, followed by genes encoding the scFv-antibody and Skp/OmpH periplasmic factor under the translational control of strong rbs from gene 10 of phage T7 (T7g10). In addition, the gene of scFv-antibody is followed by a nucleotide sequence encoding six histidine residues for both immunodetection and purification of recombinant product by immobilized metal-affinity chromatography (IMAC).

### Example 7

### Expression in Bacteria and Purification of the Multimeric Fv-Antibodies

The E. coli K12 strain RV308 (Δlac_{χ}74 galISII::OP308strA) (Maurer et al., 1980, J. Mol. Biol. 139, 147-161) (ATCC 31608) was used for functional expression of scFv-antibodies. The bacteria transformed with the expression plasmid pSKK3-scFv₇anti-CD19-SL-scFv₁₈anti-CD3 were grown overnight in 2xYT medium with 100 μg/ml ampicillin and 100 mM glucose (2xYT_{GA}) at 28° C. The overnight culture was diluted in fresh 2xYT_{GA} medium to an optical density at 600 nm (OD₆₀₀) of 0.1, and continued to grow as flask cultures at 28° C. with vigorous shaking (180-220 rpm) until OD₆₀₀ reached 0.8. Bacteria were harvested by centrifugation at 5,000 g for 15 min at 20° C., and resuspended in the same volume of fresh YTBS medium (2xYT containing 1 M sorbitol, 2.5 mM glycine betaine and 100 μg/ml ampicillin). Isopropyl-β-D-thiogalactopyranoside (IPTG) was added to a final concentration of 0.2 mM, and growth was continued at 21° C. for 18-20 h. Cells were harvested by centrifugation at 9,000 g for 20 min at 4° C. To isolate soluble periplasmic proteins, the pelleted bacteria were resuspended in 5% of the initial volume of ice-cold 200 mM Tris-HCl, 20% sucrose, 1 mM EDTA, pH 8.0. After 1 h incubation on ice with occasional stirring, the spheroplasts were centrifuged at 30,000 g for 30 min at 4° C. leaving the soluble periplasmic extract as the supernatant and spheroplasts plus the insoluble periplasmic material as the pellet. The periplasmic extract was thoroughly dialyzed against 50 mM Tris-HCl, 1 M NaCl, pH 7.0, and used as a starting material for isolating scFv-antibodies. The recombinant product was concentrated by ammonium sulfate precipitation (final concentration 70% of saturation). The protein precipitate was collected by centrifugation (10,000 g, 4° C., 40 min) and dissolved in 2.5% of the initial volume of 50 mM Tris-HCl, 1 M NaCl, pH 7.0, followed by thorough dialysis against the same buffer. Immobilized metal affinity chromatography (IMAC) was performed at 4° C. using a 5 ml column of Chelating Sepharose (Amersham Pharmacia, Freiburg, Germany) charged with Cu²⁺ and equilibrated with 50 mM Tris-HCl, 1 M NaCl, pH 7.0 (start buffer). The sample was loaded by passing the sample over the column by gravity flow. The column was then washed with twenty column volumes of start buffer followed by start buffer containing 50 mM imidazole until the absorbance (280 nm) of the effluent was minimal (about thirty column volumes). Absorbed material was eluted with 50 mM Tris-HCl, 1 M NaCl, 300 mM imidazole, pH 7.0, as 1 ml fractions. The eluted fractions containing recombinant protein were identified by Western-blot analysis using Anti-penta-His mAb (QIAGEN, Hilden, Germany) and goat anti-mouse IgG HRP-conjugated antibodies (Dianova, Hamburg, Germany) as previously described (Kipriyanov et al., 1994, Mol. Immunol. 31, 1047-1058). The positive fractions were pooled and subjected to buffer exchange for 50 mM imidazole-HCl, 50 mM NaCl (pH 6.0) using pre-packed PD-10 columns (Pharmacia Biotech, Freiburg, Germany). The turbidity of protein solution was removed by centrifugation (30,000 g, 1 h, 4° C.).

The final purification was achieved by ion-exchange chromatography on a Mono S HR 5/5 column (Amersham Biosciences, Freiburg, Germany) in 50 mM imidazole-HCl, 50 mM NaCl, pH 6.0, with a linear 0.05-1 M NaCl gradient. The fractions containing multimeric Fv-antibodies were concentrated with simultaneous buffer exchange for PBS containing 50 mM imidazole, pH 7.0 (PBSI buffer), using an Ultrafree-15 centrifugal filter device (Millipore, Eschborn, Germany). Protein concentrations were determined by the Bradford dye-binding assay (Bradford, 1976, Anal. Biochem., 72, 248-254) using the Bio-Rad (Munich, Germany) protein assay kit. SDS-PAGE analysis demonstrated that Db19-SL-scFv3 migrated as single band with a molecular mass (Mᵣ) around 56 kDa (FIG. 17). Size-exclusion chromatography on a calibrated Superdex 200 HR 10/30 column (Amersham Biosciences, Freiburg, Germany) demonstrated that Db19-SL-scFv3 was mainly in a dimeric form with Mᵣ around 150 kDa(FIG. 18).

### Example 8

### Cell Binding Measurements

The human CD3⁺ T-cell leukemia cell line Jurkat and human CD19⁺ B-cell cell line JOK-1 were used for flow cytometry experiments. The cells were cultured in RPMI 1640 medium supplemented with 10% heat-inactivated fetal calf serum (FCS), 2 mM L-glutamine, 100 U/mL penicillin G sodium and 100 μg/ml streptomycin sulfate (all from Invitrogen, Groningen, The Netherlands) at 37° C. in a humidified atmosphere with 5% CO₂. 1×10⁶ cells were incubated with 0.1 ml phosphate buffered saline (PBS, Invitrogen, Groningen, The Netherlands) supplemented with 2% heat-inactivated fetal calf serum (FCS, Invitrogen, Groningen, The Netherlands) and 0.1% sodium azide (Roth, Karlsruhe, Germany) (referred to as FACS buffer) containing diluted Db19-SL-scFv3 for 45 min on ice. After washing with FACS buffer, the cells were incubated with 0.1 ml of 0.01 mg/ml anti-(His)₆ mouse mAb 13/45/31-2 (Dianova, Hamburg, Germany) in the same buffer for 45 min on ice. After a second washing cycle, the cells were incubated with 0.1 ml of 0.015 mg/ml FITC-conjugated goat anti-mouse IgG (Dianova, Hamburg, Germany) under the same conditions as before. The cells were then washed again and resuspended in 0.5 ml of FACS buffer containing 2 μg/ml propidium iodide (Sigma-Aldrich, Taufkirchen, Germany) to exclude dead cells. The fluorescence of 1×10⁴ stained cells was measured using a Beckman-Coulter Epics XL flow cytometer (Beckman-Coulter, Krefeld, Germany). Mean fluorescence (F) was calculated using System-II and Expo32 software (Beckman-Coulter, Krefeld, Germany) and the background fluorescence was subtracted. Equilibrium dissociation constants (K_{d}) were determined by fitting the experimental values to the Lineweaver-Burk equation: 1/F=1/Fₘₐₓ+(K_{d}/Fₘₐₓ) (1/[Ab]) using the software program PRISM (GraphPad Software, San Diego, Calif.).

The flow cytometry experiments demonstrated a specific interaction of Db19-SL-scFv3 molecule to Jurkat cells expressing CD3 on their surface and to JOK-1 cells expressing CD19 on their surface (FIG. 19, A and B). The measured affinity constants proved to be fairly comparable for both CD3 and CD19-binding parts of the molecule (Table 2).

TABLE 2
Affinity of Db19-SL-scFv3 multimeric antibody binding to CD3⁺
Jurkat cells and CD19⁺ JOK-1 cells

| Cell line | K_{d} (nM) |
|---|---|
| Jurkat (CD3⁺) | 14.67 |
| JOK-1 (CD19⁺) | 10.02 |

The dissociation constants (K_{d}) were deduced from Lineweaver-Burk plots shown in FIG. 19.

### Example 9

### In Vitro Analysis of Anti-Tumor Activity of Recombinant Multivalent Molecules

Freshly isolated peripheral blood mononuclear cells (PBMC) from a patient with chronic lymphocytic leukemia (CLL) were seeded in individual wells of a 12-well plate in 2 ml RPMI-Medium/10% FCS (Invitrogen, Breda, The Netherlands) at a density of 2×10⁶ cells/ml. The recombinant antibodies Db19-SL-scFv3 were added at concentration of 5, 1, 0.1 μg/ml. After 6 days incubation, the cells were harvested, counted, and stained with anti-CD3 MAb OKT3 (DKFZ, Heidelberg, Germany), anti-CD4 MAb Edu-2 (Chemicon, Hofheim, Germany), anti-CD8 MAb UCH-T4 (Chemicon, Hofheim, Germany), and anti-CD19 MAb HD37 (DKFZ, Heidelberg, Germany) for flow cytometric analysis. 10⁴ living cells were analyzed using a Beckman-Coulter flow cytometer, and the relative amounts of CD3⁺, CD4⁺, CD8⁺ and CD19⁺ cells were plotted.

The results, shown in FIG. 20, demonstrated that tetravalent Db19-SL-scFv3 molecule caused vigorous proliferation of autologous T cells and killing of C19⁺ tumor cells. The observed T cell proliferation and killing CLL cells was even higher than those observed for previously described CD19×CD3 tandem diabody (Tandab; Kipriyanov et al. 1999, J. Mol. Biol. 293, 41-56; Cochlovius et al. 2000, Cancer Res. 60, 4336-4341).

### Example 10

### Construction of the Plasmid pSKK3-scFv_{L7}Anti-CD19-L6-scFv_{L10}anti-CD3 for the Expression of Multimeric Fv-Antibody (Db19-L6-scFv3) in Bacteria

For constructing the gene encoding the anti-CD3 scFV_{L10}, the plasmid pHOG21-dmOKT3 containing the gene for anti-human CD3 scFv₁₈ (Kipriyanov et al., 1997, Protein Engineering 10, 445-453) was used. To facilitate the cloning procedures, a NotI restriction site was introduced into the plasmid pHOG21-dmOKT3 by PCR amplification of scFv₁₈ gene using primers Bi3sk, 5′-CAGCCGGCCATGGCGCAGGTGCAACTGCAGCAG (SEQ ID NO:29) and Bi9sk, 5′-GAAGATGGATCCAGCGGCCGCAGTATCAGCCCGGTT (SEQ ID NO:30). The resulting 776 bp PCR fragment was digested with NcoI and NotI, and cloned into the NcoI/NotI-linearized vector pHOG21-CD19 (Kipriyanov et al., 1996, J. Immunol. Methods 196, 51-62), thus generating the plasmid pHOG21-dmOKT3+Not. The gene coding for OKT3 V_{H} domain with a Cys-Ser substitution at position 100A according to Kabat numbering scheme (Kipriyanov et al., 1997, Protein Engineering 10, 445-453) was amplified by PCR with primers DP1, 5′-TCACACAGAATTCTTAGATCTATTAAAGAGGAGAAATTAACC(SEQ ID NO:31) and DP2, 5′-AGCACACGATATCACCGCCAAGCTTGGGTGTTGTTTTGGC (SEQ ID NO:32), to generate the gene for anti-CD3 V_{H} followed by linker of 10 amino acids Ser-Ala-Lys-Thr-Thr-Pro-Lys-Leu-Gly-Gly. The resulting 507 bp PCR fragment was digested with NcoI and EcoRV, and cloned into NcoI/EcoRV-linearized plasmid pHOG21-dmOKT3+Not, thus generating the plasmid pHOG21-scFv10/anti-CD3.

To generate a gene encoding the anti-CD19 scFV_{L7} with the V_{L}-V_{H} orientation followed by 6 amino acid linker peptide Ser-Ala-Lys-Thr-Thr-Pro, the V_{L}-HD37 gene was amplified by PCR using as a template the plasmid DNA pHOG_HD37 (Kipriyanov et al., 1996, J. Immunol. Meth. 196, 51-62) and primers VL_Nco, 5′-CAGCCGGCCATGGCGGATATCTTGCTCACCCAAACTCCAGC and 3_Ck, 5′-AGACGGTGCAGCAACAGTACGTTTGATTTCCAGC. The resulting 371 bp PCR fragment codes for the anti-CD19 VL domain followed by 7 amino acid Arg-Thr-Val-Ala-Ala-Pro-Ser linker. In turn, the V_{H}-HD37 gene was amplified by PCR using as a template the plasmid DNA pHOG_HD37 (Kipriyanov et al., 1996, J. Immunol. Meth. 196, 51-62) and primers 5_Ck, 5′-CGTACTGTTGCTGCACCGTCTCAGGTGCAACTGCAGCAGTC and VH-L6_Pvu, 5′-CTGCTGCAGCTGCACCTGGGGTGTTGTTTTGGCTGAGGAG (SEQ ID NO:33). The resulting 428 bp PCR fragment codes for the anti-CD19 VH domain preceded by 7 amino acid Arg-Thr-Val-Ala-Ala-Pro-Ser linker and followed by 6 amino acid linker peptide Ser-Ala-Lys-Thr-Thr-Pro. The whole gene for anti-CD19 scFv_{L7}-L₆ was assembled by PCR from 371 bp and 428 bp DNA fragments using primers VL_Nco and VH-L6_Pvu. The resulting 790 bp PCR fragment was digested with NcoI and PvuII and cloned into NcoI/PvuII-linearized plasmid pHOG21-scFv10/anti-CD3, thus generating the plasmid pDISC-scFv_{L7}anti-CD19-L6-scFv_{L10}anti-CD3.

To increase the yield of functional scFv-antibodies in the bacterial periplasm, the plasmid pDISC-scFv_{L7}anti-CD19-L6-scFv_{L10}anti-CD3 was digested with NcoI and XbaI, and the 1503 bp DNA fragment was isolated and cloned into NcoI/XbaI-linearized vector pSKK3 (see Example 6). The generated plasmid pSKK3-scFv_{L7}anti-CD19-L6-scFv_{L10}anti-CD3 (FIG. 22) is suitable for expression of functional bivalent product in the E. coli periplasm under conditions of both shake-flask cultivation and high cell density fermentation.

### Example 11

### Characterization of Db19-L6-scFv3 Antibody

The recombinant scFv-antibody Db19-L6-scFv3 was expressed in E. coli RV308 cells transformed with the plasmid pSKK3-scFv_{L7}anti-CD19-L6-scFv_{L10}anti-CD3 and purified from soluble periplasmic fraction essentially as described in Example 7. SDS-PAGE analysis demonstrated that Db19-L6-scFv3 migrated as single band with a molecular mass (Mᵣ) around 56 kDa (FIG. 24). Size-exclusion chromatography on a calibrated Superdex 200 HR 10/30 column (Amersham Biosciences, Freiburg, Germany) demonstrated that Db19-L6-scFv3 was mainly in a dimeric form with Mᵣ around 150 kDa (FIG. 25).

The human CD3⁺ T-cell leukemia cell line Jurkat and human CD19⁺ B-cell cell line JOK-1 were used for flow cytometry experiments. The cells were cultured in RPMI 1640 medium supplemented with 10% heat-inactivated fetal calf serum (FCS), 2 mM L-glutamine, 100 U/mL penicillin G sodium, and 100 μg/ml streptomycin sulfate (all from Invitrogen, Groningen, The Netherlands) at 37° C. in a humidified atmosphere with 5% CO₂. 1×10⁶ cells were incubated with 0.1 ml phosphate buffered saline (PBS, Invitrogen, Groningen, The Netherlands) supplemented with 2% heat-inactivated fetal calf serum (FCS, Invitrogen, Groningen, The Netherlands) and 0.1% sodium azide (Roth, Karlsruhe, Germany) (referred to as FACS buffer) containing diluted Db19-SL-scFv3 for 45 min on ice. After washing with FACS buffer, the cells were incubated with 0.1 ml of 0.01 mg/ml anti-(His)₆ mouse mAb 13/45/31-2 (Dianova, Hamburg, Germany) in the same buffer for 45 min on ice. After a second washing cycle, the cells were incubated with 0.1 ml of 0.015 mg/ml FITC-conjugated goat anti-mouse IgG (Dianova, Hamburg, Germany) under the same conditions as before. The cells were then washed again and resuspended in 0.5 ml of FACS buffer containing 2 μg/ml propidium iodide (Sigma-Aldrich, Taufkirchen, Germany) to exclude dead cells. The fluorescence of 1×10⁴ stained cells was measured using a Beckman-Coulter Epics XL flow cytometer (Beckman-Coulter, Krefeld, Germany). Mean fluorescence (F) was calculated using System-II and Expo32 software (Beckman-Coulter, Krefeld, Germany), and the background fluorescence was subtracted. Equilibrium dissociation constants (K_{d}) were determined by fitting the experimental values to the Lineweaver-Burk equation: 1/F=1/Fₘₐₓ+(K_{d}/Fₘₐₓ) (1/[Ab]) using the software program PRISM (GraphPad Software, San Diego, Calif.).

The flow cytometry experiments demonstrated a specific interaction of Db19-L6-scFv3 molecule to Jurkat cells expressing CD3 on their surface and to JOK-1 cells expressing CD19 on their surface (FIGS. 26,A and B). The measured affinity constants proved to be fairly comparable for both CD3 and CD19-binding parts of the molecule (Table 3).

TABLE 3
Affinity of Db19-L6-scFv3 multimeric antibody binding to CD3⁺
Jurkat cells and CD19⁺ JOK-1 cells

| Cell line | K_{d} (nM) |
|---|---|
| Jurkat (CD3⁺) | 4.42 |
| JOK-1 (CD19⁺) | 8.49 |

The dissociation constants (K_{d}) were deduced from Lineweaver-Burk plots shown in FIG. 26.

1. A multimeric structure comprising two or more identical protein monomers, characterized by the following features:
(a) the monomers of said structure comprise at least four variable domains of which the first or last two variable domains of which the first or last two variable domains form an antigen-binding VH-VL or VL-VH scFv unit wherein two variable domains are linked by a peptide linker of at least 5 amino acid residues which does not prevent the intramolecular formation of a scFv;
(b) the other two neighboring variable domains of the monomer are non-covalently bound to the complementary domains of another monomer resulting in the formation of at least two additional antigen binding sites to form the multimerisation motif.

2. The multimeric structure of claim 1, in form of a multimeric Fv-antibody, having the following features:
(a) the monomers of said Fv-antibody comprise at least four variable domains of which the first or last two variable domains are linked by a peptide linker of 5 to 30 acid residues, which does not prevent the intramolecular formation of a scFv.
(b) the other two neighboring variable domains of the monomer are non-covalently bound to two complementary variable domains of another monomer resulting in the formation of at least two additional antigen binding sites to form a multimerization motif, wherein said two variable domains are linked by a peptide linker of a maximum of 12 amino aid residues.

3. The multimeric Fv-antibody of claim 2, wherein a further feature is that the antigen-binding V_{H}-V_{L} or V_{L}-V_{H} scFv unit formed by the two neighboring domains of one monomer is linked to the other variable domains of the multimerization motif by a peptide linker of 5 to 30 amino acid residues.

4. The mutlimeric structure of claim 1, wherein said monomers comprise four variable domains and wherein the third and fourth variable domains of said one end of the monomers are linked by a peptide linker, said peptide linker having 12 or less amino acid residues.

5. The mutlimeric structure of claim 1, wherein said monomers comprise four variable domains and wherein the first and second variable domains of said one end of the monomers are linked by a peptide linker, said peptide linker having 12 or less amino residues.

6. The multimeric structure of claim 2, wherein the second and third variable domain of the monomers are linked by a peptide linker consisting of 5 to 30 amino acid residues.

7. The multimeric structure of claim 1, wherein any variable domain of the monomers is shortened by at least one amino acid residue at their N- and/or C-terminus.

8. The multimeric structure of claim 1, wherein the order of domains of a monomer is V_{H}-V_{L}-V_{H}-V_{L}, V_{L}-V_{H}-V_{H}-V_{L}, V_{H}-V_{L}-V_{L}-V_{H} or V_{L}-V_{H}-V_{L}-V_{H}.

9. The multimeric structure of claim 1, wherein the non-covalent binding of at least one pair of variable domains is strengthened by at least one disulfide bridge.

10. The multimeric structure of claim 1, which is a tetravalent dimer, hexavalent trimer or octavalent tetramer.

11. The multimeric structure of claim 1, which is a bisepcific, of trispecific or tetraspecific antibody.

12. The multimeric structure of claim 1, wherein at least one monomer is linked to a biologically active substance, a chemical agent, a peptide, a protein or a drug.

13. The multimeric structure of claim 1, which is a monospecific antibody capable of specifically binding the CD19 antigen of B-lymphocytes or the CEA antigen.

14. The multimeric structure of claim 1, which is a bispecific antibody capable of specifically bi9dning:
(a) CD19 and the CD3 complex of the T-cell receptor;
(b) CD19 and the CD5 complex of the T-cell receptor;
(c) CD19 and the CD28 antigen on T-lymphocytes;
(d) CD19 and the CD16 on natural killer cells, macrophages and activated monocytes;
(e) CEA and CD3;
(f) CEA and CE28; or
(g) CEA and CDE16.

15. A process for the preparation of a multimeric structure of claim 1, wherein (a) DNA sequences encoding the peptide linkers are ligated with the DNA sequences encoding the variable domains such that the peptide linkers connect the variable domains resulting in the formation of a DNA sequence encoding a monomer of the multivalent multimeric structure and (b) the DNA sequences encoding the various monomers are expressed in a suitable expression system.

16. A DNA sequence encoding a multimeric structure of claim 1.

17. An expression vector containing the DNA sequence of claim 16.

18. The expression vector of claim 17, which is pSKK2-scFv_{L18}anti-CD3-LL-scFv_{L10}anti-CD19 (pSKK2-scFv3LL Db19) (DSM 14470) or psKK2-scFv_{L18}antiCD19-LL-scFv_{L10}anti-CD3(pSKK2-scFv19LL Db3) (DSM 14471).

19. A host cell containing the expression vector of claim 17.

20. A pharmaceutical composition comprising a dimeric or multimeric structure of claim 1.

21. Use of a dimeric or multimeric structure of claim 1 for diagnosis.

22. Use of a dimeric or multimeric structure of claim 1 for the preparation of a pharmaceutical composition for (a) the treatment of a viral, bacterial, tumoral or prion related disease, (b) the agglutination of red blood cells, (c) linking cytotoxic cells of the immune system to tumor cells, or (d) linking activating cytokines, cytotoxic substances or a protease to a target cell.

23. A diagnostic kit comprising a multimeric structure of claim 1.

24. A pharmaceutical composition comprising a DNA sequence of claim 17.

25. A pharmaceutical composition comprising an expression vector of claim 18.
